SEQUENCE LISTING (114 sequences)

SEQ ID NO: 1 - DNA, 348 bp, Mus sp.
gaggtgcagctgcaggagtctggaggtgagctggtaaggcctgggacttcagtgaagatatcctgcaaggcttctggctacaccttcactaacttctggctaggttggataaaggagaggcctggacatggacttgagtggattggagacatttaccctggaggtcattatactaactacaatgagaacttcaagggcaaggccacactgactgcagacacatcctccaacactgcctacatgcaagtcagtagcctgacatctgaggactctgctgtctatttctgttttactcaatactatgatatggactactggggtcaaggaacctcagtcaccgtctcctca

SEQ ID NO: 2 - protein, 116 aa, Mus sp.
EVQLQESGGELVRPGTSVKISCKASGYTFTNFWLGWIKERPGHGLEWIGDIYPGGHYTNYNENFKGKATLTADTSSNTAYMQVSSLTSEDSAVYFCFTQYYDMDYWGQGTSVTVSS

SEQ ID NO: 3 - protein, 8 aa, Mus sp.
GYTFTNFW

SEQ ID NO: 4 - protein, 8 aa, Mus sp.
IYPGGHYT

SEQ ID NO: 5 - protein, 9 aa, Mus sp.
FTQYYDMDY

SEQ ID NO: 6 - DNA, 339 bp, Mus sp.
gatgttgtgatgacccagactccactcactttgtcggttaccattggacaaccagcctccatctcttgcaagtcaagtcagagcctcttatatagtgatggagagacatatttgaattggttgttacagaggccaggccagtctccaaagcgcctaatctatctggtgtctaaactggactctggagtccctgacaggttcactggcagtggatcagggacagatttcacactgaaaattagcagagtggaggctgaggatttgggagtttattattgctggcaaggtacacactttcctcagacgttcggtggaggcaccaagctggaaatcaaacgg

SEQ ID NO: 7 - protein, 113 aa, Mus sp.
DVVMTQTPLTLSVTIGQPASISCKSSQSLLYSDGETYLNWLLQRPGQSPKRLIYLVSKLDSGVPDRFTGSGSGTDFTLKISRVEAEDLGVYYCWQGTHFPQTFGGGTKLEIKR

SEQ ID NO: 8 - protein, 11 aa, Mus sp.
QSLLYSDGETY

SEQ ID NO: 9 - protein, 3 aa, Mus sp.
LVS

SEQ ID NO: 10 - protein, 9 aa, Mus sp.
WQGTHFPQT

SEQ ID NO: 11 - DNA, 345 bp, Mus sp.
gaggtgcagctgcaggagtctggacctgagctggtgaagcctggggcttcagtgaagatgtcctgtaaggcttctggatacacattcactgactactacatgaactgggtgaagcagagccatggaaagagccttgagtggattggagatattaatccttacaacggtgatactacctacaaccagaagttcaagggcaaggccacattgactgtagacaaatcctccagcacagcctacatgcagctcaacagcctgacatctgaggactctgcagtctattactgtgcaagtgggtacgagggtgactactggggccaaggcaccactctcacagtctcctca

SEQ ID NO: 12 - protein, 115 aa, Mus sp.
EVQLQESGPELVKPGASVKMSCKASGYTFTDYYMNWVKQSHGKSLEWIGDINPYNGDTTYNQKFKGKATLTVDKSSSTAYMQLNSLTSEDSAVYYCASGYEGDYWGQGTTLTVSS

SEQ ID NO: 13 - protein, 8 aa, Mus sp.
GYTFTDYY

SEQ ID NO: 14 - protein, 8 aa, Mus sp.
INPYNGDT

SEQ ID NO: 15 - protein, 8 aa, Mus sp.
ASGYEGDY

SEQ ID NO: 16 - DNA, 336 bp, Mus sp.
gatattgtgatgacgcaggctgcattctccaatccagtcactcttggaacatcagcttccatttcctgcaggtctagtaagagtctcctacatagtaatggcatcacttatttgtattggtatctgcagaagccaggccagtctcctcrgctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctacgttcggatcggggaccaagctggaaatcaaacgt

SEQ ID NO: 17 - protein, 112 aa, Mus sp.
  MOD_RES (50)..(50): Any amino acid
DIVMTQAAFSNPVTLGTSASISCRSSKSLLHSNGITYLYWYLQKPGQSPXLLIYQMSNLASGVPDRFSSSGSGTDFTLRISRVEAEDVGVYYCAQNLELPTFGSGTKLEIKR

SEQ ID NO: 18 - protein, 11 aa, Mus sp.
KSLLHSNGITY

SEQ ID NO: 19 - protein, 3 aa, Mus sp.
QMS

SEQ ID NO: 20 - protein, 8 aa, Mus sp.
AQNLELPT

SEQ ID NO: 21 - DNA, 351 bp, Mus sp.
gaggtgcagctgcaggagtctgggggagacttagtgaagcctggagggtccctgaaactctcctgtgcagcctctggattcactttcagtagctataccatgtcttgggttcgccagactccggagaggaggctggaatgggtcgcaaccattactagtggtggtacttacacctactatccagacagtgtgaagggccgattcaccatctccagagacaatgccaagaacaccctgtacctgcaaatgaccagtctgaagtctgaggacacagccatgtattactgtacaagagaggatggttactacttttcttactggggccaagggactctggtcactgtctctgca

SEQ ID NO: 22 - protein, 117 aa, Mus sp.
EVQLQESGGDLVKPGGSLKLSCAASGFTFSSYTMSWVRQTPERRLEWVATITSGGTYTYYPDSVKGRFTISRDNAKNTLYLQMTSLKSEDTAMYYCTREDGYYFSYWGQGTLVTVSA

SEQ ID NO: 23 - protein, 8 aa, Mus sp.
GFTFSSYT

SEQ ID NO: 24 - protein, 8 aa, Mus sp.
ITSGGTYT

SEQ ID NO: 25 - protein, 10 aa, Mus sp.
TREDGYYFSY

SEQ ID NO: 26 - DNA, 339 bp, Mus sp.
gatgttgtgatgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaagatttgggagtttattactgcgtgcaaggtacacattttccgtacacgttcggaggggggaccaagctggaaataaaacgg

SEQ ID NO: 27 - protein, 113 aa, Mus sp.
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPYTFGGGTKLEIKR

SEQ ID NO: 28 - protein, 11 aa, Mus sp.
QSLLYSNGKTY

SEQ ID NO: 29 - protein, 3 aa, Mus sp.
LVS

SEQ ID NO: 30 - protein, 9 aa, Mus sp.
VQGTHFPYT

SEQ ID NO: 31 - DNA, 369 bp, Mus sp.
gaggtgcagctgcaggagtctggacctggactggtgaagcctggggcttcagtgaagatatcctgcaaggcttctggttactcattcactggctactacatgaattgggtgaagcaaagtcctgaaaagagccttgagtggattggagagattaatcctagcactggtggtactacctacaaccagaagttcaaggccaaggccacattgactgtagacaaatcctccagcacagcctacatgcagctcaagagcctgacatctgaggactctgcagtctattactgtgtgagatattcccattactacggtagtggctcctggtacttcgatgtctggggcacagggaccacggtcaccgtctcctca

SEQ ID NO: 32 - protein, 123 aa, Mus sp.
EVQLQESGPGLVKPGASVKISCKASGYSFTGYYMNWVKQSPEKSLEWIGEINPSTGGTTYNQKFKAKATLTVDKSSSTAYMQLKSLTSEDSAVYYCVRYSHYYGSGSWYFDVWGTGTTVTVSS

SEQ ID NO: 33 - protein, 8 aa, Mus sp.
GYSFTGYY

SEQ ID NO: 34 - protein, 8 aa, Mus sp.
INPSTGGT

SEQ ID NO: 35 - protein, 16 aa, Mus sp.
VRYSHYYGSGSWYFDV

SEQ ID NO: 36 - DNA, 324 bp, Mus sp.
gacatccagatgactcagtctccagcctccctatctgcatctgtgggagaaactgtcaccatcacatgtcgagcaagtgaaaatatttacagtaatttagcatggtatcagcagaaacagggaaaatctcctcagctcctggtcaatgctgcaacaaatttagcagatggtgtgccatcaaggttcagtggcagtggatcaggcacacagttttctctgaagatcaacagcctgcagcctgaagattttgggagttattactgtcaacatttttatgttactccgtatatgttcggatcggggaccaagctggaaataaaacgg

SEQ ID NO: 37 - protein, 108 aa, Mus sp.
DIQMTQSPASLSASVGETVTITCRASENIYSNLAWYQQKQGKSPQLLVNAATNLADGVPSRFSGSGSGTQFSLKINSLQPEDFGSYYCQHFYVTPYMFGSGTKLEIKR

SEQ ID NO: 38 - protein, 6 aa, Mus sp.
ENIYSN

SEQ ID NO: 39 - protein, 3 aa, Mus sp.
AAT

SEQ ID NO: 40 - protein, 9 aa, Mus sp.
QHFYVTPYM

SEQ ID NO: 41 - DNA, 363 bp, Mus sp.
gaggtgcagctgcaggagtctggacctggcctagtgcagccctcacagagcctgtccatcacctgcacagtctctggtttctcattaactagctatggtgtacactgggttcgccagtctccaggaaagggtctggagtggctgggagtcatatggagtggtggaagcacagactataatgcagctttcatatccagactgagcatcagcaaggacaattccaagagccaagttttctttaaaatgaacagtctgcaagctgatgacacagccatatattactgtgccagaaattggtttgattacgacaattactattctatggactactggggtcaaggaacctcagtcaccgtctcctca

SEQ ID NO: 42 - protein, 121 aa, Mus sp.
EVQLQESGPGLVQPSQSLSITCTVSGFSLTSYGVHWVRQSPGKGLEWLGVIWSGGSTDYNAAFISRLSISKDNSKSQVFFKMNSLQADDTAIYYCARNWFDYDNYYSMDYWGQGTSVTVSS

SEQ ID NO: 43 - protein, 8 aa, Mus sp.
GFSLTSYG

SEQ ID NO: 44 - protein, 7 aa, Mus sp.
IWSGGST

SEQ ID NO: 45 - protein, 15 aa, Mus sp.
ARNWFDYDNYYSMDY

SEQ ID NO: 46 - DNA, 324 bp, Mus sp.
gatatccagatgacacagactacatcctccctgtctgcctctctgggagacagagtcaccatcagttgcagggcaagtcaggatattagcaattatttaaactggtatcagcagaaaccagatggaactgttaaactcctgatctactacacatcaagattacattcaggagtcccatcaaggttcagtggcagtgggtctgggacagatttttctctcaccatcagcaaccaggaacctgaagatattgccacttactattgtcagcagtatagtaaccttccgtggacgttcggtggaggcaccaagttggaaatcaaacgg

SEQ ID NO: 47 - protein, 108 aa, Mus sp.
DIQMTQTTSSLSASLGDRVTISCRASQDISNYLNWYQQKPDGTVKLLIYYTSRLHSGVPSRFSGSGSGTDFSLTISNQEPEDIATYYCQQYSNLPWTFGGGTKLEIKR

SEQ ID NO: 48 - protein, 6 aa, Mus sp.
QDISNY

SEQ ID NO: 49 - protein, 3 aa, Mus sp.
YTS

SEQ ID NO: 50 - protein, 9 aa, Mus sp.
QQYSNLPWT

SEQ ID NO: 51 - DNA, 354 bp, Mus sp.
gaggtgcagctgcaggagtctggacctgagctgaagaagcctggagagacagtcaagatctcctgcaaggcttctgggtataccttcacaacctatggaatgagctgggtgaaacaggctccaggaaagggtttaaagtggatgggccggataaacacctactctggagtgccaacatatgctgatgagttcaagggacggtttgccttctctttggaaaattctgccagcactgcctatttgcagatcaacaacctcaaaaatgaggacacggcgacatatttctgtgcaaggggcgatggtggccactggtttgctaactggggccaagggactctggtcactgtctctgca

SEQ ID NO: 52 - protein, 118 aa, Mus sp.
EVQLQESGPELKKPGETVKISCKASGYTFTTYGMSWVKQAPGKGLKWMGRINTYSGVPTYADEFKGRFAFSLENSASTAYLQINNLKNEDTATYFCARGDGGHWFANWGQGTLVTVSA

SEQ ID NO: 53 - protein, 8 aa, Mus sp.
GYTFTTYG

SEQ ID NO: 54 - protein, 8 aa, Mus sp.
INTYSGVP

SEQ ID NO: 55 - protein, 11 aa, Mus sp.
ARGDGGHWFAN

SEQ ID NO: 56 - DNA, 324 bp, Mus sp.
gacatccagatgacacagtctccatcctcactgtctgcatctctgggaggcaaagtcaccatcacttgcaaggcaagccaagacattaacaagaatatagcttggtaccaacacaagcctggaaaaggtcctaggctgctcatatggtacaaatctgcattacagccaggcatcccatcaaggttcagtggaagtgggtctgggagagattattccttcagcatcagcaacctggagcctgaagatattgcaacttattcctgtctacagtatgataatcttccaccgacgttcggtggaggcaccaagctggaaatcaaacgg

SEQ ID NO: 57 - protein, 108 aa, Mus sp.
DIQMTQSPSSLSASLGGKVTITCKASQDINKNIAWYQHKPGKGPRLLIWYKSALQPGIPSRFSGSGSGRDYSFSISNLEPEDIATYSCLQYDNLPPTFGGGTKLEIKR

SEQ ID NO: 58 - protein, 6 aa, Mus sp.
QDINKN

SEQ ID NO: 59 - protein, 3 aa, Mus sp.
YKS

SEQ ID NO: 60 - protein, 9 aa, Mus sp.
LQYDNLPPT

SEQ ID NO: 61 - DNA, 357 bp, Mus sp.
gaggtgcagctgcaggagtctggccctgggatattgcagccctcccagaccctcagtctgacttgttctttctctgggttttcactgagaactcttggtatgggtgtaggctggattcgtcagtcttcagggaagggtctggagtggctggcacacatttggtggagtgatgagaattactataatccagccctgaagagtcggctcacaatctccaaggatacctccaaaaaccaggtattcctcaggatcgccaatgtggacactgcagaaactgccacatactactgtgctcgaatagcctatgatggttactttgactactggggccaaggcaccagtctcacagtctcctca

SEQ ID NO: 62 - protein, 119 aa, Mus sp.
EVQLQESGPGILQPSQTLSLTCSFSGFSLRTLGMGVGWIRQSSGKGLEWLAHIWWSDENYYNPALKSRLTISKDTSKNQVFLRIANVDTAETATYYCARIAYDGYFDYWGQGTSLTVSS

SEQ ID NO: 63 - protein, 10 aa, Mus sp.
GFSLRTLGMG

SEQ ID NO: 64 - protein, 7 aa, Mus sp.
IWWSDEN

SEQ ID NO: 65 - protein, 12 aa, Mus sp.
CARIAYDGYFDY

SEQ ID NO: 66 - DNA, 348 bp, Mus sp.
atgtcaggtcacagcagaaacatgaagtttccttctcaacttctgctcttactgctgtttggaatcccaggagacagagtcaccattacttgcaaggcaagtgaggacatatataatcggttagcctggtatcagcagaaaccaggaaatgctcctagactcttaatatctggtgcaaccagtttggaaactggggttccttcaagattcagtggcagtggatctggaaaggattacactctcagcattaccagtcttcagactgaagatgttgctacttattactgtcaacaatattggactactccgtggacgttcggtggaggcaccaagctggaaataaaacgt

SEQ ID NO: 67 - protein, 116 aa, Mus sp.
MSGHSRNMKFPSQLLLLLLFGIPGDRVTITCKASEDIYNRLAWYQQKPGNAPRLLISGATSLETGVPSRFSGSGSGKDYTLSITSLQTEDVATYYCQQYWTTPWTFGGGTKLEIKR

SEQ ID NO: 68 - protein, 6 aa, Mus sp.
EDIYNR

SEQ ID NO: 69 - protein, 3 aa, Mus sp.
GAT

SEQ ID NO: 70 - protein, 9 aa, Mus sp.
QQYWTTPWT

SEQ ID NO: 71 - DNA, 345 bp, Mus sp.
gaggtgcagctgcaggagtctggagctgagctggtgaggcctggggcttcagtgacgctgtcctgcaagacttcgggctacacatttactgactatgaaatgcactgggtgaagcagacacctgtgcatggcctggaatggattggagctattgatcctgaaactggtggtactgcctacaatcagaagttcaagggcaaggccacactgactgcagacaaatcctccagcacagcctacatggagctcagccgcctgacatctgaggactctgccgtctattactgtacgggtaactatgctacggactactggggtcaaggaacctcagtcaccgtctcctca

SEQ ID NO: 72 - protein, 115 aa, Mus sp.
EVQLQESGAELVRPGASVTLSCKTSGYTFTDYEMHWVKQTPVHGLEWIGAIDPETGGTAYNQKFKGKATLTADKSSSTAYMELSRLTSEDSAVYYCTGNYATDYWGQGTSVTVSS

SEQ ID NO: 73 - protein, 8 aa, Mus sp.
GYTFTDYE

SEQ ID NO: 74 - protein, 8 aa, Mus sp.
IDPETGGT

SEQ ID NO: 75 - protein, 8 aa, Mus sp.
TGNYATDY

SEQ ID NO: 76 - DNA, 339 bp, Mus sp.
gacattgtgatgtcacagtctccatcctccctggctgtgtcagcaggagagaaggtcactatgagctgcaaatccagtcagagtctgttcaacagtagaagtcgaaagaactacttggcttggtaccagctgaaaccaggacagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatctaattatctgtggacgttcggtggaggcaccaagctggaaatcaaacgg

SEQ ID NO: 77 - protein, 113 aa, Mus sp.
DIVMSQSPSSLAVSAGEKVTMSCKSSQSLFNSRSRKNYLAWYQLKPGQSPKLLIYWASTRESGVPDRFTGSGSGTDFTLTISSVQAEDLAVYYCKQSNYLWTFGGGTKLEIKR

SEQ ID NO: 78 - protein, 12 aa, Mus sp.
QSLFNSRSRKNY

SEQ ID NO: 79 - protein, 3 aa, Mus sp.
WAS

SEQ ID NO: 80 - protein, 8 aa, Mus sp.
KQSNYLWT

SEQ ID NO: 81 - DNA, 369 bp, Mus sp.
gaggtgcagctgcaggagtctggacctgagctggtgaagcctggggcttcagtgaagatatcctgcaaggcttctggttactcattcactggctactacatgaattgggtgaagcaaagtcctgaaaagagccttgagtggattggagagattaatcctagcactggtggtactacctacaaccagaagttcaaggccaaggccacattgactgtagacaaatcctccagcacagcctacatgcagctcaagagcctgacatctgaggactctgcagtctattactgtgtgagatattcccattactacggtagtggctcctggtacttcgatgtctggggcacagggaccacggtcaccgtctcctca

SEQ ID NO: 82 - protein, 123 aa, Mus sp.
EVQLQESGPELVKPGASVKISCKASGYSFTGYYMNWVKQSPEKSLEWIGEINPSTGGTTYNQKFKAKATLTVDKSSSTAYMQLKSLTSEDSAVYYCVRYSHYYGSGSWYFDVWGTGTTVTVSS

SEQ ID NO: 83 - protein, 8 aa, Mus sp.
GYSFTGYY

SEQ ID NO: 84 - protein, 8 aa, Mus sp.
INPSTGGT

SEQ ID NO: 85 - protein, 16 aa, Mus sp.
VRYSHYYGSGSWYFDV

SEQ ID NO: 86 - DNA, 324 bp, Mus sp.
gacatccagatgactcagtctccagcctccctatctgcatctgtgggagaaactgtcaccatcacatgtcgagcaagtgagaatatttacagtaacttagcatggtatcagcagaaacagggaaaatctcctcagctcctggtcaatgctgcaacaaatttagcagatggtgtgccatcaaggttcagtggcagtggatcaggcacacagttttctctgaagatcaacagcctgcagcctgaagattttgggagttattactgtcaacatttttatgttactccgtatacgttcggatcggggaccaagctggaaataaaacgg

SEQ ID NO: 87 - protein, 108 aa, Mus sp.
DIQMTQSPASLSASVGETVTITCRASENIYSNLAWYQQKQGKSPQLLVNAATNLADGVPSRFSGSGSGTQFSLKINSLQPEDFGSYYCQHFYVTPYTFGSGTKLEIKR

SEQ ID NO: 88 - protein, 6 aa, Mus sp.
ENIYSN

SEQ ID NO: 89 - protein, 3 aa, Mus sp.
AAT

SEQ ID NO: 90 - protein, 9 aa, Mus sp.
QHFYVTPYT

SEQ ID NO: 91 - protein, 151 aa, Marburg marburgvirus
SGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRH

SEQ ID NO: 92 - protein, 148 aa, Sudan ebolavirus
KDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIRE

SEQ ID NO: 93 - protein, 148 aa, Zaire ebolavirus
RDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLRE

SEQ ID NO: 94 - protein, 15 aa, Marburg marburgvirus
DSPLEASKRWAFRTG

SEQ ID NO: 95 - protein, 15 aa, Marburg marburgvirus
EASKRWAFRTGVPPK

SEQ ID NO: 96 - protein, 15 aa, Sudan ebolavirus
STDIPSATKRWGFRS

SEQ ID NO: 97 - protein, 15 aa, Zaire ebolavirus
ATDVPSATKRWGFRS

SEQ ID NO: 98 - protein, 15 aa, Zaire ebolavirus
SATKRWGFRSGVPPK

SEQ ID NO: 99 - protein, 15 aa, Marburg marburgvirus
LQKTEDVHLMGFTLS

SEQ ID NO: 100 - protein, 15 aa, Marburg marburgvirus
EDVHLMGFTLSGQKV

SEQ ID NO: 101 - protein, 15 aa, Marburg marburgvirus
LMGFTLSGQKVADSP

SEQ ID NO: 102 - protein, 15 aa, Marburg marburgvirus
TLSGQKVADSPLEAS

SEQ ID NO: 103 - protein, 15 aa, Marburg marburgvirus
QKVADSPLEASKRWA

SEQ ID NO: 104 - protein, 15 aa, Marburg marburgvirus
DSPLEASKRWAFRTG

SEQ ID NO: 105 - protein, 15 aa, Marburg marburgvirus
EASKRWAFRTGVPPK

SEQ ID NO: 106 - protein, 15 aa, Marburg marburgvirus
RWAFRTGVPPKNVEY

SEQ ID NO: 107 - protein, 15 aa, Marburg marburgvirus
RTGVPPKNVEYTEGE

SEQ ID NO: 108 - protein, 15 aa, Marburg marburgvirus
PPKNVEYTEGEEAKT

SEQ ID NO: 109 - protein, 10 aa, Artificial Sequence
  Synthetic consensus sequence derived from multiple filovirus glycoproteins
XAXKRWXFRX

SEQ ID NO: 110 - protein, 7 aa, Artificial Sequence
  Synthetic consensus sequence derived from multitple filovirus glycoproteins
RWXFRXG

SEQ ID NO: 111 - protein, 7 aa, Marburg virus
RWAFRTG

SEQ ID NO: 112 - protein, 7 aa, Ebola virus
RWGFRSG

SEQ ID NO: 113 - protein, 9 aa, Artificial Sequence
  Synthetic hemagglutinin tag derived from influenza virus HA
YPYDVPDYA

SEQ ID NO: 114 - protein, 7 aa, Artificial Sequence
  Synthetic cleavage site for factor Xa
IEGRGAR

(19) United States
(12) Patent Application Publication
(10) Pub. No.: US 2017/0158753 A1
(43) Pub. Date: Jun. 8, 2017

(54) MONOCLONAL ANTIBODIES DIRECTED AGAINST ENVELOPE GLYCOPROTEINS FROM MULTIPLE FILOVIRUS SPECIES

(71) Applicant: INTEGRATED BIOTHERAPEUTICS, INC., Rockville, MD (US)

(72) Inventors: Mohammad Javad Aman, Rockville, MD (US); Frederick W. Holtsberg, Taneytown, MD (US); Sergey Shulenin, Point of Rocks, MD (US); Hong Vu, North Potomac, MD (US); Katie A. Howell, Rockville, MD (US); Kelly Lyn Warfield, Adamstown, MD (US)

(21) Appl. No.: 15/321,833

(22) PCT Filed: Jun. 24, 2015
(86) PCT No.: PCT/US15/37493

§ 371 Date: Dec. 23, 2016

Related U.S. Application Data
(60) Provisional application No. 62/016,811, filed on Jun. 25, 2014.
(60) Provisional application No. 62/019,668, filed on Jul. 1, 2014.

(51) Int. Cl. C07K 16/10; G01N 33/569

(52) U.S. Cl. CPC C07K 16/10; G01N 33/56983; C07K 2317/56; C07K 2317/76; C07K 2317/94; C07K 2317/565; C07K 2317/31; C07K 2317/52; G01N 2333/08; C07K 2317/92; A61K 2039/505

(57) ABSTRACT

The disclosure provides binding molecules, e.g., antibodies or antigen-binding fragments thereof, that can bind to orthologous epitopes found on two or more filovirus species or strains.

### RELATED APPLICATIONS

This application claims the benefit of U.S. Provisional Applications 62/016,811, filed on Jun. 25, 2014, and 62/019,668, filed on Jul. 1, 2015, which are both incorporated by reference herein in their entireties.

### REFERENCE TO SEQUENCE LISTING SUBMITTED ELECTRONICALLY

The content of the electronically submitted sequence listing in ASCII text file (Name 57783_145911_Seq_List_ST25.txt; Size: 45,264 bytes; and Date of Creation: Jun. 23, 2015) filed with the application is incorporated herein by reference in its entirety.

### BACKGROUND

The Filoviridae family consists of a single species of Marburg virus (MARV) as well as five species of ebolavirus: Ebola (EBOV), Sudan (SUDV), Bundibugyo (BDBV), Reston (RESTV), and Tai Forest (TAFV) viruses (Kuhn, J. H., et al., Viruses 6, 4760-4799 (2014); Kuhn, J. H., et al., Viruses 6, 3663-3682 (2014)).

Filoviruses cause lethal hemorrhagic fever in humans and non-human primates with case fatality rates of up to 90% (Feldmann, H. & Kiley, M. P., Curr Top Microbiol Immunol 235, 1-21 (1999); Feldmann, H. & Klenk, H. D., Adv Virus Res 47, 1-52 (1996)). EBOV has caused the majority of Ebola virus disease (EVD) outbreaks including the 2014 outbreak in West Africa with over 27000 cases and 11,000 death (Gire, S. K., et al., Science 345, 1369-1372 (2014)). However, other members of Filoviridae have also caused sizable human outbreaks including seven outbreaks of SUDV, two outbreaks of BDBV, and twelve outbreaks of MARV (Chippaux, J. P., J Venom Anim Toxins Incl Trop Dis 20, 44 (2014); Pigott, D. M., et al., Trans R Soc Trop Med Hyg 109(6), 366 (2015)). RESTV has not caused disease in humans but its recent detection in pigs has raised concern about the potential emergence of ebolaviruses in human food chain (Barrette, R. W., et al., Science 325, 204-206 (2009)). Thus there is urgent need for development of broadly protective countermeasures for filoviruses as the nature of future outbreaks cannot be predicted.

It is well established that the filovirus glycoproteins represent the primary protective antigens (Feldmann, et al., 2003, Nat Rev Immunol, 3 (8):677-685; Feldmann, et al., 2005, Curr Opin Investig Drugs, 6 (8):823-830; Geisbert, et al., 2010, Rev Med Virol, 20(6):344-57). GP consists of a receptor binding GP1 subunit connected with the GP2 fusion domain via a disulfide link (See, e.g., FIG. 4A). We have previously identified a specific region of the MARV and EBOV GP1 consisting of ˜150 amino acids that binds filovirus receptor-positive cells, but not receptor-negative cells, more efficiently than GP₁, and compete with the entry of the respective viruses (Kuhn, et al., 2006, J Biol Chem, 281 (23):15951-15958). This region of GP is referred to here as receptor binding region (RBR) and is part of a larger domain that excludes the variable, glycosylated, and bulky mucin-like domain (MLD). The RBR shows the highest level of homology between Filovirus glycoproteins (Kuhn, et al., 2006).

Role of Antibodies in Protection Against Filovirus Hemorrhagic Fever.

While both T and B cell responses are reported to play a role in protective immune responses to filoviruses (Warfield, et al., 2005, J Immunol, 175 (2):1184-1191), a series of recent reports indicate that antibody alone can provide protection. Dye et al showed that purified convalescent IgG from macaques can protect non-human primates (NHPs) against challenge with MARV and EBOV when administered as late as 48 h post exposure (Dye, et al., 2012, Proc Natl Acad Sci USA, 109(13):5034-9). Olinger et al. reported protection from EBOV challenge in NHPs treated with a cocktail of three monoclonal antibodies (mAbs) to GP administered 24 h and 48 h post exposure (Olinger, et al., 2012, Proc Natl Acad Sci USA, 109 (44):18030-18035). Similar results were also reported in two other studies (Qiu, et al., 2013, Sci Transl Med, 5 (207):207ra143; Qiu, et al., 2013, J Virol, 87 (13):7754-7757). Collectively these data demonstrate that a humoral response can control, alleviate, reduce, or prevent, filovirus infection.

Recent reports indicate that monoclonal antibodies (mAbs) against the filovirus glycoproteins (GP) represent effective post-exposure treatments for Marburg and Ebola hemorrhagic fever. See, e.g., Qiu, X., et al., Nature 514(7520), 47 (2014). However, all previously reported ebolavirus antibodies are species-specific and the majority of them target EBOV (Zaire). The primary sequence of GP shows 56-65% identity between the various ebolavirus species while the sequence identity between Ebola and Marburg GP is less than 30%. Despite this homology no cross reactive antibodies have been described so far that would protect against multiple filovirus species.

The 2014 outbreak of EVD in West Africa has highlighted the threat of filoviruses to global health (Baize, S. Curr Opin Virol 10, 70-76 (2015)) and there is also growing concern that aerosolized filoviruses can be used as biowarfare agents Marzi, A. & Feldmann, H., Expert Rev Vaccines 13, 521-531 (2014); Salvaggio, M. R. & Baddley, J. W., Dermatol Clin 22, 291-302, vi (2004); Kuhn, J. H., et al., Biosecur Bioterror 9, 361-371 (2011).). While significant progress has been made towards vaccines and immunotherapeutics for EBOV (Zaire), the development of therapeutics against other filovirus species has been lagging behind. Broadly protective treatment modalities are urgently needed as the nature of future outbreaks cannot be predicted.

### SUMMARY

This disclosure provides an isolated binding molecule or antigen-binding fragment thereof that includes a first binding domain that specifically binds to an orthologous filovirus glycoprotein epitope, where the binding domain specifically binds to the epitope on two or more filovirus species or strains, for example, in two or more, three or more, four or more, or five or more of Marburg virus (MARV), Ravn virus (RAVV), Tai Forest virus (TAFV), Reston virus (RESTV), Sudan virus (SUDV), Ebola virus (EBOV), and Bundibugyo virus (BDBV). In certain aspects the first binding domain can bind to the orthologous epitope as expressed in two or more, three or more, four or more, or all five of EBOV, SUDV, MARV, RESTV, and BDBV. In certain aspects the orthologous epitope is in the receptor-binding region (RBR) of GP-1 subunit of the viral glycoprotein.

In certain aspects the first binding domain can bind to the orthologous epitope as expressed in EBOV, SUDV, MARV, RESTV, and BDBV. In certain aspects the first binding domain can bind to the same orthologous epitope as an antibody or antigen-binding fragment thereof including a heavy chain variable region (VH) and light chain variable region (VL) including, respectively, the amino acid sequences SEQ ID NO: 2 and 7, or SEQ ID NO: 12 and 17, or can competitively inhibit antigen binding by an antibody or antigen-binding fragment thereof including a heavy chain variable region (VH) and light chain variable region (VL) including, respectively, the amino acid sequences SEQ ID NO: 2 and 7, or SEQ ID NO: 12 and 17. In certain aspects first binding domain can bind to an orthologous epitope within the amino acid consensus sequence of S/E-A-S/T-K-R-W-A/G-F-R-T/S (SEQ ID NO: 109), or the amino acid consensus sequence R-W-A/G-F-R-T/S-G (SEQ ID NO: 110).

In certain aspects the first binding domain can bind to the orthologous epitope as expressed in at least EBOV, SUDV, and MARV. In certain aspects the first binding domain can bind to the same orthologous epitope as an antibody or antigen-binding fragment thereof including a VH and a VL including the amino acid sequences SEQ ID NO: 22 and 27, or can competitively inhibit antigen binding by an antibody or antigen-binding fragment thereof including a VH and a VL including the amino acid sequences SEQ ID NO: 22 and 27.

In certain aspects the first binding domain can bind to the orthologous epitope as expressed in EBOV, SUDV, RESTV, and BDBV. In certain aspects the first binding domain can bind to the same orthologous epitope as an antibody or antigen-binding fragment thereof including a VH and a VL including, respectively, the amino acid sequences SEQ ID NO: 32 and 37, SEQ ID NO: 42 and 47, or SEQ ID NO: 62 and 67, or can competitively inhibit antigen binding by an antibody or antigen-binding fragment thereof including a VH and a VL including, respectively, the amino acid sequences SEQ ID NO: 32 and 37, SEQ ID NO: 42 and 47, or SEQ ID NO: 62 and 67.

In certain aspects the first binding domain can bind to the orthologous epitope as expressed in EBOV, SUDV, and BDBV. In certain aspects the first binding domain can bind to the same orthologous epitope as an antibody or antigen-binding fragment thereof including a VH and a VL including the amino acid sequences SEQ ID NO: 52 and 57, or can competitively inhibit antigen binding by an antibody or antigen-binding fragment thereof including a VH and a VL including the amino acid sequences SEQ ID NO: 52 and 57.

In certain aspects the first binding domain can bind to the orthologous epitope as expressed in SUDV and MARV. In certain aspects the first binding domain can bind to the same orthologous epitope as an antibody or antigen-binding fragment thereof including a VH and a VL including the amino acid sequences SEQ ID NO: 72 and 77, or can competitively inhibit antigen binding by an antibody or antigen-binding fragment thereof including a VH and a VL including the amino acid sequences SEQ ID NO: 72 and 77.

In certain aspects the first binding domain can bind to the orthologous epitope as expressed in EBOV, SUDV, and RESTV. In certain aspects the first binding domain can bind to the same orthologous epitope as an antibody or antigen-binding fragment thereof including a VH and a VL including the amino acid sequences SEQ ID NO: 82 and 87, or can competitively inhibit antigen binding by an antibody or antigen-binding fragment thereof including a VH and a VL including the amino acid sequences SEQ ID NO: 82 and 87.

In certain aspects the first binding domain can bind to the orthologous epitope in solution at a pH of about 4.0, about 4.5, about 5.0, about 5.5, about 6.0, about 6.5, about 7.0, or about 7.5.

In certain aspects the binding molecule or fragment thereof of any one of claims 1 to 31, which includes an antibody or antigen-binding fragment thereof where the first binding domain includes VL-CDR1, VL-CDR2, VL-CDR3, VH-CDR1, VH-CDR2, and VH-CDR3 amino acid sequences identical or identical except for four, three, two, or one single amino acid substitutions, deletions, or insertions in one or more CDRs to: SEQ ID NOs: 3, 4, 5, 8, 9, and 10; SEQ ID NOs: 13, 14, 15, 18, 19, and 20; SEQ ID NOs: 23, 24, 25, 28, 29, and 30; SEQ ID NOs: 33, 34, 35, 38, 39, and 40; SEQ ID NOs: 43, 44, 45, 48, 49, and 50; SEQ ID NOs: 53, 54, 55, 58, 59, and 60; SEQ ID NOs: 63, 64, 65, 68, 69, and 70; SEQ ID NOs: 73, 74, 75, 78, 79, and 80; or SEQ ID NOs: 83, 84, 85, 88, 89, and 90; respectively. In certain aspects the first binding domain includes VH and VL amino acid sequences at least 85%, 90%, 95%, or 100% identical to reference amino acid sequences SEQ ID NO: 2 and SEQ ID NO: 7; SEQ ID NO: 12 and SEQ ID NO: 17; SEQ ID NO: 22 and SEQ ID NO: 27; SEQ ID NO: 32 and SEQ ID NO: 37; SEQ ID NO: 42 and SEQ ID NO: 47; SEQ ID NO: 52 and SEQ ID NO: 57; SEQ ID NO: 62 and SEQ ID NO: 67; SEQ ID NO: 72 and SEQ ID NO: 77; or SEQ ID NO: 82 and SEQ ID NO: 87; respectively. The antibody can be a human antibody, a murine antibody, a humanized antibody, a chimeric antibody, or a fragment thereof, and/or can be a monoclonal antibody, a component of a polyclonal antibody mixture, a recombinant antibody, a multispecific antibody, or any combination thereof. In certain aspects the antibody or fragment thereof is a bispecific antibody or fragment thereof further including a second binding domain. In certain aspects the second binding domain can specifically bind to a filovirus epitope that is surface exposed and accessible to the second binding domain on a filovirus virion particle. In certain aspects the second binding domain can specifically bind to the mucin-like domain, an epitope located in the glycan cap, an epitope located in the GP2 fusion domain, or any combination thereof.

In certain aspects binding of the first binding domain to the orthologous epitope on a filovirus fully or partially neutralizes infectivity of the filovirus.

The disclosure further provides a composition including the antibody or fragment thereof of any one of claims 32 to 54, and a carrier, and a kit, including the antibody or antigen binding fragment thereof or composition as provided herein, and instructions for using the antibody or fragment thereof or using the composition or directions for obtaining instructions for using the antibody or fragment thereof or using the composition.

The disclosure further provides an isolated polynucleotide including a nucleic acid encoding the binding molecule or fragment thereof as provided herein or a subunit thereof, or the antibody or fragment thereof as provided herein; or a subunit thereof. Also provided are vectors comprising one or more polynucleotides as provided, and a host cell including the polynucleotide or combination of polynucleotides as provided or the vector or vectors as provided. The disclosure further provides a method of making the binding molecule or fragment thereof of or the antibody or fragment thereof as provided where the method includes culturing the provided host cell; and isolating the binding molecule or fragment thereof or antibody or fragment thereof.

The disclosure further provides a method for preventing, treating, or managing filovirus infection in a subject, where the method includes administering to a subject in need thereof an effective amount of the antibody or antigen binding fragment thereof as provided herein.

The disclosure further provides a method of neutralizing a virus that enters host cells through fusion events in the host cell endosomes, including contacting the virus with a bispecific antibody including a first binding domain and a second binding domain, where the first binding domain binds to a epitope of the virus that interacts with a host cell surface receptor, and the second binding domain binds to an epitope on the surface of the intact virus, where the virus/antibody complex is inhibited from fusing with the host cell membrane in the endosome, thereby neutralizing the virus.

### BRIEF DESCRIPTION OF THE DRAWINGS/FIGURES

FIG. 1 A-C: Immunization design and serology results for generation of pan-filovirus antibodies. A. Immunization schedules. B. Antibody responses to EBOV, SUDV, and MARV in the pooled sera from the terminal bleeds of Groups 1 and 2. C. Percent neutralization of VSV-GP pseudotyped viruses for EBOV, SUDV, and MARV by 1:10 dilution of terminal sera from Group 1.

FIG. 2: Binding profile of pan-ebolavirus mAbs. ELISA plates were coated with GPΔTM from the indicated filovirus species. The binding of the mAbs was tested over a wide range of concentrations as described in the Online Methods. Data are shown as optical density (OD₆₅₀) for m16G8 (A), m8C4 (B), m17C6 (C), m4B8 (D), and m21D10 (E) graphed against concentration of mAbs in nanomolar (nM).

FIG. 3A-B: Reactivity of pan filovirus mAbs m21D10 (panel A) and m2D8 (panel B) to filovirus glycoproteins using Western blot analysis. Glycoproteins used were either the full ectodomain (GPΔTM) or MLD-deleted GP (GPΔmuc). GPs from different species are identified as S (SUDV), E (EBOV), M (MARV), B (BDBV), and R (RESTV).

FIG. 4A-E: Binding region of the pan-ebolavirus mAbs. (A) Schematic of the domain organization of EBOV GP and structure of the various truncation proteins used for domain mapping. Dose dependent binding of each mAb to GPΔTM (B), GPΔmuc (C), thermolysin cleaved GP (GPcl) (D), and soluble GP (sGP) (E) is shown as determined by ELISA.

FIG. 5A-B: Competition of binding of pan filovirus mAb m21D10 to filovirus glycoproteins by linear peptides corresponding to sequences within receptor binding region. A) ELISA plates were coated with GPΔmuc proteins from the three major filovirus species as indicated in the figure. MAb m21D10 at a concentration of 10 μg/ml was incubated with 8 μg/ml of specific peptides S-7 (SEQ ID NO: 96), Z-16 (SEQ ID NO: 97), Z-17 (SEQ ID NO: 98), M-16 (SEQ ID NO: 94), M-17 (SEQ ID NO: 95), an irrelevant peptide, or PBS alone. The mixtures were added to the coated plates, incubated for an hour, washed, and bound antibody was determined using a secondary antibody against mouse IgG and detected by TMB substrate. B) Alignment of the sequence of the specific peptides tested in panel A and secondary structure of the corresponding region from Zaire GP structure. Dotted box shows the shared sequence of m21D10 epitope.

FIG. 6: MLD and glycan cap restrict access of m21D10 to its epitope. (A) dose-dependent binding of m21D10 to GPΔTM, GPΔmuc, and GPcl determined by ELISA. (B) Binding of m21D10 to GPΔTm and GPΔmuc of SUDV and MARV.

FIG. 7: Competition of binding of pan filovirus mAb m2D8 to Marburg virus glycoproteins by linear peptides corresponding to sequences within receptor binding region (SEQ ID Nos: 99-108, respectively). ELISA plates were coated with MARV Musoke GPΔmuc protein. The binding sequences derived from the competing peptides are shown in bold.

FIG. 8A-C: Neutralizing activity of the pan-ebolavirus mAbs against EBOV and SUDV. (A) Percent neutralization by m8C4 of vesicular stomatitis virus (VSV) pseudotyped with EBOV or SUDV GP. (B and C) Neutralizing activity of the pan-ebolavirus antibodies against wild type SUDV (B) and EBOV (C) determined using a high content-imaging based neutralization assay.

FIG. 9A-E: Protection of mice from lethal challenge by pan-ebolavirus antibodies. (A, B) groups of 15 mice or 5 mice (for m4B8) were infected with 1000 PFU of MA-EBOV and treated intraperitoneally with 25 mg/kg of the indicated mAb 2 hours or 3 days post infection. (C, D) Efficacy of m4B8 (30 mg/kg) treatment on day 3 only. (E, F) Efficacy of combination of m8C4 and m16G8 (15 mg/kg) treatment on days 0 and 3 or day 3 only.

FIG. 10A-B: Efficacy of m8C4 against Sudan virus infection. Groups of ten IFNαβ^{-/-} mice were infected with wild type SUDV. Two groups of mice received m8C4 either once on day 1 at 5 mg/kg or on days −1, +1, +3 relative to infection at a dose of 10 mg/kg. Control mice received PBS only. Survival (A) and weight change (B) were monitored and recorded.

FIG. 11A-C: Schematic of a prototypic BETAb construct (A), filovirus glycoprotein (B), and blockade of the receptor binding region (RBR) upon entry of virus-BETAb complex into the endosomes (C). Abbreviations: VL: variable region of immunoglobulin light chain; CL: constant region of immunoglobulin light chain, VH: variable region of immunoglobulin heavy chain; CH1-CH3: constant regions of immunoglobulin heavy chain; GP2: glycoprotein fusion domain; MLD: mucin-like domain.

FIG. 12: Binding of pan filovirus monoclonal antibodies m2D8 (Panel A) and m21D10 (Panel B) to EBOV GPΔmuc) and cathepsin cleaved EBOV GP (containing RBR and GP2) at neutral and acidic pH determined by ELISA.

### DETAILED DESCRIPTION

### Definitions

The term “a” or “an” entity refers to one or more of that entity; for example, “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, deletions or additions. Derivatives are polypeptides that have been altered so as to exhibit additional features not found on the native polypeptide. Examples include fusion proteins. Variant polypeptides can also be referred to herein as “polypeptide analogs.” As used herein a “derivative” refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

Disclosed herein are certain binding molecules, or antigen-binding fragments, variants, or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is a bispecific antibody or fragment thereof that comprises at least two distinct binding domains that specifically bind different antigenic determinants or epitopes. In certain aspects, a bispecific antibody as provided herein can be said to comprise a first binding domain binding to a first epitope, and a second binding domain binding to a second epitope.

The terms “antibody” and “immunoglobulin” can be used interchangeably herein. An antibody (or a fragment, variant, or derivative thereof as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. This quaternary binding molecule structure forms the antigen-binding site present at the end of each arm of the Y. More specifically, the antigen-binding site is defined by three CDRs on each of the VH and VL chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acids when compared against each other. Nevertheless, application of either definition to refer to a CDR of an antibody or variants thereof is intended to be within the scope of the term as defined and used herein. The appropriate amino acids that encompass the CDRs as defined by each of the above-cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Immunoglobulin variable domains can also be analyzed using the IMGT information system (www://imgt.cines.fr/) (IMGT®/V-Quest) to identify variable region segments, including CDRs. See, e.g., Brochet, X. et al., Nucl. Acids Res. 36:W503-508 (2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid positions in a binding molecule which specifically binds to a filovirus glycoprotein subunit, e.g, an antibody, or antigen-binding fragment, variant, or derivative thereof as disclosed herein are according to the Kabat numbering system.

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen, e.g., a filovirus glycoprotein subunit disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. A binding molecule as disclosed herein can be said to bind a target antigen, e.g., a filovirus glycoprotein subunit, with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen, e.g., a filovirus glycoprotein subunit with an on rate (k(on)) of greater than or equal to 10³ M⁻¹ sec⁻¹, 5×10³M⁻¹ sec⁻¹, 10⁴ M⁻¹ sec⁻¹ or 5×10⁴ M⁻¹ sec⁻¹. A binding molecule as disclosed herein can be said to bind a target antigen, e.g., a filovirus glycoprotein subunit with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof can be said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross-reactive if it binds to an epitope other than the one that induced its formation, e.g., various different filovirus receptor binding regions. The cross-reactive epitope contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10¹³ M, 10⁻¹³ M, 5×10⁻¹⁴ M, 10⁻¹⁴ M, 5×10⁻¹⁵ M, or 10⁻¹⁵ M.

Antibody fragments including single-chain antibodies can comprise the variable region(s) alone or in combination with the entirety or a portion of the following: hinge region, CH1, CH2, and CH3 domains. Also included are antigen-binding fragments that comprise any combination of variable region(s) with a hinge region, CH1, CH2, and CH3 domains. Binding molecules, e.g., antibodies, or antigen-binding fragments thereof disclosed herein can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain, a binding molecule, e.g., an antibody comprising a heavy chain portion comprises at least one of: a CH1 domain, a hinge (e.g., upper, middle, and/or lower hinge region) domain, a CH2 domain, a CH3 domain, or a variant or fragment thereof. For example, a binding molecule, e.g., an antibody or fragment, variant, or derivative thereof can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a binding molecule, e.g., an antibody or fragment, variant, or derivative thereof comprises a polypeptide chain comprising a CH3 domain. Further, a binding molecule for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

The heavy chain portions of a binding molecule, e.g., an antibody as disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. The light chain portion comprises at least one of a VL or CL domain.

Binding molecules, e.g., antibodies or antigen-binding fragments, variants, or derivatives thereof disclosed herein can be described or specified in terms of the epitope(s) or portion(s) of an antigen, e.g., a target a filovirus glycoprotein subunit that they recognize or specifically bind. The portion of a target antigen that specifically interacts with the antigen-binding domain of an antibody is an “epitope,” or an “antigenic determinant.” A target antigen, e.g., a filovirus glycoprotein subunit can comprise a single epitope, but typically comprises at least two epitopes, and can include any number of epitopes, depending on the size, conformation, and type of antigen. As used herein, an “orthologous epitope” refers to versions of an epitope found in related organisms, e.g., different filovirus species. Orthologous epitopes can be similar in structure, but can vary in one or more amino acids.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about amino acid 244 to amino acid 360 of an antibody using conventional numbering schemes (amino acids 244 to 360, Kabat numbering system; and amino acids 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 amino acids.

As used herein, the term “hinge region” includes the portion of a heavy chain molecule that joins the CH1 domain to the CH2 domain. This hinge region comprises approximately 25 amino acids and is flexible, thus allowing the two N-terminal antigen-binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The term “bispecific antibody” as used herein refers to an antibody that has binding sites for two different antigens within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more CDRs from an antibody of known specificity and, by partial framework region replacement and sequence changing. Although the CDRs can be derived from an antibody of the same class or even subclass as the antibody from which the framework regions are derived, it is envisaged that the CDRs will be derived from an antibody of different class, e.g., from an antibody from a different species. An engineered antibody in which one or more “donor” CDRs from a non-human antibody of known specificity is grafted into a human heavy or light chain framework region is referred to herein as a “humanized antibody.” In some instances only those amino acids that are necessary to maintain the activity of the target-binding site are transferred. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “coding region” is a portion of nucleic acid comprising codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association or linkage can be when a coding region for a gene product, e.g., a polypeptide, can be associated with one or more regulatory sequences in such a way as to place expression of the gene product under the influence or control of the regulatory sequence(s). Two DNA fragments (such as a polypeptide coding region and a promoter associated therewith) can be “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA).

Polynucleotide and nucleic acid coding regions can be associated with additional coding regions that encode secretory or signal peptides, which direct the secretion of a polypeptide encoded by a polynucleotide as disclosed herein, e.g., a polynucleotide encoding a polypeptide subunit provided herein. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art.

A “transformed” cell, or a “host” cell, is a cell into which a nucleic acid molecule has been introduced by molecular biology techniques. As used herein, the term transformation encompasses all techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the terms “treat,” “treatment,” or “treatment of” (e.g., in the phrase “treating a subject”) refers to reducing the potential for disease pathology, reducing the occurrence of disease symptoms, e.g., to an extent that the subject has a longer survival rate or reduced discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals, including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective, and that contains no additional components that are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

Certain therapies can provide “synergy” and prove “synergistic”, i.e., an effect can be achieved when the active ingredients are used together that is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

Pan-Filovirus Binding Molecules

This disclosure provides a pan-filovirus binding molecule, e.g., a cross-reactive anti-filovirus antibody or antigen-binding fragment thereof. Pan-filovirus binding molecules can be useful for treatment of a filovirus infection without it being necessary to know the exact filovirus species or strain. More specifically, the disclosure provides an isolated binding molecule or antigen-binding fragment thereof comprising a first binding domain that specifically binds to an orthologous filovirus glycoprotein epitope, wherein the binding domain specifically binds to the epitope on two, three, four, five, or more filovirus species or strains. In certain aspects the pan-filovirus binding molecule can be a cross-reactive antibody or antigen-binding fragment thereof. In certain aspects the binding molecule can be a bispecific antibody that can facilitate targeting of the binding molecule to the endosomal region of a filovirus-infected cell, e.g., through a second binding domain.

In certain aspects, the first binding domain of a pan-filovirus binding molecule, e.g., a cross-reactive anti-filovirus antibody or antigen-binding fragment thereof can specifically bind to a filovirus orthologous epitope as expressed in two or more, three or more, four or more, or five or more filovirus species including, Marburg virus (MARV), Ravn virus (RAVV), Tai Forest virus (TAFV), Reston virus (RESTV), Sudan virus (SUDV), Ebola virus (EBOV), and Bundibugyo virus (BDBV). For example, the first binding domain of a pan-filovirus binding molecule, e.g., a cross-reactive anti-filovirus antibody or antigen-binding fragment thereof can bind to an orthologous filovirus epitope as expressed in two or more, three or more, four or more, or five of EBOV, SUDV, MARV, RESTV, and BDBV. Any filovirus epitope which has similarities across filovirus species can be a target of the first binding domain of a pan-filovirus binding molecule as provided herein. In certain aspects, the orthologous epitope can be in the receptor-binding region (RBR) of GP-1 subunit of the viral glycoprotein.

Two exemplary first binding domains can be derived from the VH and VL antigen binding domains of murine monoclonal antibodies m2D8 and m21D10, which bind to the RBR across at least five different species of filovirus, e.g., the first binding domain can bind to the orthologous epitope as expressed in EBOV, SUDV, MARV, RESTV, and BDBV. In certain aspects the first binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 2 and 7 (the VH and VL of m2D8, which binds to an orthologous epitope within the amino acid sequence generically depicted as SEQ ID NO: 109), or SEQ ID NO: 12 and 17 (the VH and VL of m21D10, which binds to an orthologous epitope within the amino acid sequence generically depicted as SEQ ID NO: 110). In certain aspects the first binding domain of this exemplary pan-filovirus binding molecule or fragment thereof can competitively inhibit antigen binding by an antibody or antigen-binding fragment thereof comprising a VH and a VL comprising, respectively, the amino acid sequences SEQ ID NO: 2 and 7, or SEQ ID NO: 12 and 17.

Another exemplary first binding domain can be derived from the VH and VL antigen binding domains of murine monoclonal antibody m5E4, which can bind to the filovirus glycoprotein across at least three species of filovirus, e.g., the first binding domain can bind to the orthologous epitope as expressed in at least EBOV, SUDV, MARV. In certain aspects the first binding domain of this exemplary pan-filovirus binding molecule or fragment thereof can bind to the same orthologous epitope as an antibody or antigen-binding fragment thereof comprising a VH and a VL comprising, respectively, the amino acid sequences SEQ ID NO: 22 and 27 (the VH and VL of m5E4). In certain aspects the first binding domain of this exemplary pan-filovirus binding molecule or fragment thereof can competitively inhibit antigen binding by an antibody or antigen-binding fragment thereof comprising a VH and a VL comprising, respectively, the amino acid sequences SEQ ID NO: 22 and 27.

Another exemplary first binding domain can be derived from the VH and VL antigen binding domains of murine monoclonal antibody m16G8, m17C6, or m4B8, each of which can bind to the filovirus glycoprotein across four species of filovirus, e.g., the first binding domain can bind to the orthologous epitope as expressed in EBOV, SUDV, RESTV, and BDBV. In certain aspects the first binding domain of this exemplary pan-filovirus binding molecule or fragment thereof can bind to the same orthologous epitope as an antibody or antigen-binding fragment thereof comprising a VH and a VL comprising, respectively, the amino acid sequences SEQ ID NO: 32 and 37 (the VH and VL of m16G8), SEQ ID NO: 42 and 47 (the VH and VL of m17C6), or SEQ ID NO: 62 and 67 (the VH and VL of m4B8). In certain aspects the first binding domain of this exemplary pan-filovirus binding molecule or fragment thereof can competitively inhibit antigen binding by an antibody or antigen-binding fragment thereof comprising a VH and a VL comprising the amino acid sequences SEQ ID NO: 32 and 37, SEQ ID NO: 42 and 47, or SEQ ID NO: 62 and 67, respectively.

Another exemplary first binding domain can be derived from the VH and VL antigen binding domains of murine monoclonal antibody m8C4, which can bind to the filovirus glycoprotein across two species of filovirus, e.g., the first binding domain can bind to the orthologous epitope as expressed in EBOV and SUDV. In certain aspects the first binding domain of this exemplary pan-filovirus binding molecule or fragment thereof can bind to the same orthologous epitope as an antibody or antigen-binding fragment thereof comprising a VH and a VL comprising, respectively, the amino acid sequences SEQ ID NO: 52 and 57 (the VH and VL of m8C4). In certain aspects the first binding domain of this exemplary pan-filovirus binding molecule or fragment thereof can competitively inhibit antigen binding by an antibody or antigen-binding fragment thereof comprising a VH and a VL comprising, respectively, the amino acid sequences SEQ ID NO: 52 and 57.

Another exemplary first binding domain can be derived from the VH and VL antigen binding domains of murine monoclonal antibody m21B2, which can bind to the filovirus glycoprotein across at least two species of filovirus, e.g., the first binding domain can bind to the orthologous epitope at least as expressed in SUDV and MARV. In certain aspects the first binding domain of this exemplary pan-filovirus binding molecule or fragment thereof can bind to the same orthologous epitope as an antibody or antigen-binding fragment thereof comprising a VH and a VL comprising, respectively, the amino acid sequences SEQ ID NO: 72 and 77 (the VH and VL of m21B2). In certain aspects the first binding domain of this exemplary pan-filovirus binding molecule or fragment thereof can competitively inhibit antigen binding by an antibody or antigen-binding fragment thereof comprising a VH and a VL comprising, respectively, the amino acid sequences SEQ ID NO: 42 and 47.

Another exemplary first binding domain can be derived from the VH and VL antigen binding domains of murine monoclonal antibody m2E4, which can bind to the filovirus glycoprotein across three species of filovirus, e.g., the first binding domain can bind to the orthologous epitope as expressed in EBOV, SUDV, and RESTV. In certain aspects the first binding domain of this exemplary pan-filovirus binding molecule or fragment thereof can bind to the same orthologous epitope as an antibody or antigen-binding fragment thereof comprising a VH and a VL comprising, respectively, the amino acid sequences SEQ ID NO: 82 and 87 (the VH and VL of m2E4). In certain aspects the first binding domain of this exemplary pan-filovirus binding molecule or fragment thereof can competitively inhibit antigen binding by an antibody or antigen-binding fragment thereof comprising a VH and a VL comprising, respectively, the amino acid sequences SEQ ID NO: 82 and 87.

In certain aspects a binding molecule as provided herein can be capable of functioning at the pH found in endosomal compartments of filovirus infected cells, e.g., at an acidic pH For example in certain aspects a first binding domain of a binding molecule as provide herein can bind to an orthologous filovirus epitope in solution at a pH of about 4.0, about 4.5, about 5.0, about 5.5, about 6.0, about 6.5, about 7.0, or about 7.5.

In certain aspects a pan-filovirus binding molecule as provided herein can be an anti-filovirus antibody or antigen-binding fragment thereof. For example in certain aspects the disclosure provides an pan-filovirus antibody or antigen-binding fragment thereof comprising a first binding domain that comprises VL-CDR1, VL-CDR2, VL-CDR3, VH-CDR1, VH-CDR2, and VH-CDR3 amino acid sequences identical or identical except for four, three, two, or one single amino acid substitutions, deletions, or insertions in one or more CDRs to: SEQ ID NOs: 3, 4, 5, 8, 9, and 10; SEQ ID NOs: 13, 14, 15, 18, 19, and 20; SEQ ID NOs: 23, 24, 25, 28, 29, and 30; SEQ ID NOs: 33, 34, 35, 38, 39, and 40; SEQ ID NOs: 43, 44, 45, 48, 49, and 50; SEQ ID NOs: 53, 54, 55, 58, 59, and 60; SEQ ID NOs: 63, 64, 65, 68, 69, and 70; SEQ ID NOs: 73, 74, 75, 78, 79, and 80; or SEQ ID NOs: 83, 84, 85, 88, 89, and 90; respectively.

Furthermore, in certain aspects the disclosure provides an pan-filovirus antibody or antigen-binding fragment thereof comprising a first binding domain that comprises VH and VL amino acid sequences at least 85%, 90%, 95%, or 100% identical to reference amino acid sequences SEQ ID NO: 2 and SEQ ID NO: 7; SEQ ID NO: 12 and SEQ ID NO: 17; SEQ ID NO: 22 and SEQ ID NO: 27; SEQ ID NO: 32 and SEQ ID NO: 37; SEQ ID NO: 42 and SEQ ID NO: 47; SEQ ID NO: 52 and SEQ ID NO: 57; SEQ ID NO: 62 and SEQ ID NO: 67; SEQ ID NO: 72 and SEQ ID NO: 77; or SEQ ID NO: 82 and SEQ ID NO: 87; respectively.

A pan filovirus antibody or antigen-binding fragment thereof as provided herein can be, a human antibody, a murine antibody, a humanized antibody, a chimeric antibody, or a fragment thereof. Moreover, the antibody or fragment thereof can be a monoclonal antibody, a component of a polyclonal antibody mixture, a recombinant antibody, a multispecific antibody, or any combination thereof.

In certain aspects, a pan-filovirus antibody or fragment thereof as provided herein can be a bispecific antibody or fragment thereof that further comprises a second binding domain. Certain bispecific antibodies as provided herein can be engineered to be targeted to the endosomal regions of a filovirus-infected cell. For example, the second binding domain can specifically bind to a filovirus epitope that can be surface exposed and accessible to the second binding domain on a filovirus virion particle. In this aspect, the bispecific antibody can be targeted to the endosomal compartment of an infected cell, where cathepsin enzymes can cleave the mucin-like domain that masks the receptor binding region on native filovirus virion particles, thus opening the RBR up to the first binding domain which can then bind to the virus and neutralize the virus infectivity. In certain aspects, the second binding domain can bind to a surface exposed epitope on a virion particle, for example, the second binding domain can specifically bind to an epitope located in the mucin-like domain, an epitope located in the glycan cap, an epitope located in the GP2 fusion domain, or any combination thereof.

An antibody or fragment thereof of as provided herein can in certain aspects comprise a heavy chain constant region or fragment thereof. The heavy chain can be a murine constant region or fragment thereof, e.g., a human constant region or fragment thereof, e.g., IgM, IgG, IgA, IgE, IgD, or IgY constant region or fragment thereof. Various human IgG constant region subtypes or fragments thereof can also be included, e.g., a human IgG1, IgG2, IgG3, or IgG4 constant region or fragment thereof.

An antibody or fragment thereof as provided herein can further comprise a light chain constant region or fragment thereof. For example, the light chain constant region or fragment thereof can be a murine constant region or fragment thereof, e.g., a human light chain constant region or fragment thereof, e.g., a human kappa or lambda constant region or fragment thereof.

In certain aspects the first binding domain of a pan-filovirus antibody or fragment thereof as provided herein comprises a full-size antibody comprising two heavy chains and two light chains. In other aspects, the first binding domain of a pan-filovirus antibody or fragment thereof as provided herein comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain aspects the second binding domain of a pan-filovirus antibody or fragment thereof as provided herein comprises a full-size antibody comprising two heavy chains and two light chains. In other aspects, the second binding domain of a pan-filovirus antibody or fragment thereof as provided herein comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain aspects a pan-filovirus antibody or fragment thereof as provided herein fully or partially neutralizes infectivity of the filovirus upon binding of the first binding domain to the orthologous epitope on a filovirus.

In certain aspects, a pan-filovirus antibody or fragment thereof as provided herein can be conjugated to an antiviral agent, a protein, a lipid, a detectable label, a polymer, or any combination thereof.

The disclosure further provides a composition comprising a pan-filovirus binding molecule, e.g., a cross-reactive anti-filovirus antibody or antigen-binding fragment thereof, and a carrier.

Polynucleotides

In certain aspects the disclosure provides an isolated polynucleotide comprising a nucleic acid encoding a pan-filovirus binding molecule, e.g., a cross-reactive anti-filovirus antibody or antigen-binding fragment thereof or a subunit thereof. For example, a polynucleotide as provided herein can include a nucleic acid encoding a VH, wherein the VH comprises VH-CDR1, VH-CDR2, and VH-CDR3, wherein the VH-CDRs comprise, respectively, amino acid sequences identical to, or identical except for four, three, two, or one single amino acid substitutions, deletions, or insertions in one or more of the VH-CDRs to: SEQ ID NOs: 3, 4, and 5; SEQ ID NOs: 13, 14, and 15; SEQ ID NOs: 23, 24, and 25; SEQ ID NOs: 33, 34, and 35; SEQ ID NOs: 43, 44, and 45; SEQ ID NOs: 53, 54, and 55; SEQ ID NOs: 63, 64, and 65; SEQ ID NOs: 73, 74, and 75; or SEQ ID NOs: 83, 84, and 85.

Moreover, a polynucleotide as provided herein can include a nucleic acid encoding a VL that includes a VL-CDR1, a VL-CDR2, and a VL-CDR3, wherein the VL-CDRs comprise, respectively, amino acid sequences identical to, or identical except for four, three, two, or one single amino acid substitutions, deletions, or insertions in one or more of the VL-CDRs to: SEQ ID NOs: 8, 9, and 10; SEQ ID NOs: 18, 19, and 20; SEQ ID NOs: 28, 29, and 30; SEQ ID NOs: 38, 39, and 40; SEQ ID NOs: 48, 49, and 50; SEQ ID NOs: 58, 59, and 60; SEQ ID NOs: 68, 69, and 70; SEQ ID NOs: 78, 79, and 80; or SEQ ID NOs: 88, 89, and 90.

In certain aspects, a polynucleotide as provided herein an include a nucleic acid encoding a VH that comprises an amino acid sequence at least 85%, 90%, 95%, or 100% identical to the reference amino acid sequence SEQ ID NO: 2; SEQ ID NO: 12; SEQ ID NO: 22; SEQ ID NO: 32; SEQ ID NO: 42; SEQ ID NO: 52; SEQ ID NO: 62; SEQ ID NO: 72; or SEQ ID NO: 82. In certain aspects, a polynucleotide as provided herein an include a nucleic acid encoding a VL, wherein the VL comprises an amino acid sequence at least 85%, 90%, 95%, or 100% identical to the reference amino acid sequence SEQ ID NO: 7; SEQ ID NO: 17; SEQ ID NO: 27; SEQ ID NO: 37; SEQ ID NO: 47; SEQ ID NO: 57; SEQ ID NO: 67; SEQ ID NO: 77; or SEQ ID NO: 87.

The disclosure further provides a vector comprising a polynucleotide as provided herein, and a composition comprising a polynucleotide or a vector as provided herein.

In certain aspects the disclosure provides a polynucleotide or a combination of polynucleotides encoding a pan-filovirus binding molecule, e.g., a cross-reactive anti-filovirus antibody or antigen-binding fragment thereof. In certain aspects the polynucleotide or combination of polynucleotides can comprise a nucleic acid encoding a VH, and a nucleic acid encoding a VL, wherein the VH and VL comprise VL-CDR1, VL-CDR2, VL-CDR3, VH-CDR1, VH-CDR2, and VH-CDR3 amino acid sequences identical or identical except for four, three, two, or one single amino acid substitutions, deletions, or insertions in one or more CDRs to: SEQ ID NOs: 3, 4, 5, 8, 9, and 10; SEQ ID NOs: 13, 14, 15, 18, 19, and 20; SEQ ID NOs: 23, 24, 25, 28, 29, and 30; SEQ ID NOs: 33, 34, 35, 38, 39, and 40; SEQ ID NOs: 43, 44, 45, 48, 49, and 50; SEQ ID NOs: 53, 54, 55, 58, 59, and 60; SEQ ID NOs: 63, 64, 65, 68, 69, and 70; SEQ ID NOs: 73, 74, 75, 78, 79, and 80; or SEQ ID NOs: 83, 84, 85, 88, 89, and 90; respectively.

In certain aspects the polynucleotide or combination of polynucleotides can comprise a nucleic acid encoding a VH, and a nucleic acid encoding a VL, wherein the VH and VL comprise amino acid sequences at least 85%, 90%, 95%, or 100% identical to reference amino acid sequences selected from the group consisting of SEQ ID NO: 2 and SEQ ID NO: 7; SEQ ID NO: 12 and SEQ ID NO: 17; SEQ ID NO: 22 and SEQ ID NO: 27; SEQ ID NO: 32 and SEQ ID NO: 37; SEQ ID NO: 42 and SEQ ID NO: 47; SEQ ID NO: 52 and SEQ ID NO: 57; SEQ ID NO: 62 and SEQ ID NO: 67; SEQ ID NO: 72 and SEQ ID NO: 77; or SEQ ID NO: 82 and SEQ ID NO: 87; respectively.

In certain aspects of the polynucleotide or combination of polynucleotides as provided herein the nucleic acid encoding a VH and the nucleic acid encoding a VL can be in the same vector. Such a vector is also provided.

In certain aspects of the polynucleotide or combination of polynucleotides as provided herein the nucleic acid encoding a VH and the nucleic acid encoding a VL can be in different vectors. Such vectors are further provided.

The disclosure also provides a host cell comprising the polynucleotide or combination of polynucleotides as provided herein or the vector or vectors as provided.

Moreover, the disclosure provides a method of making a pan-filovirus binding molecule, e.g., a cross-reactive anti-filovirus antibody or antigen-binding fragment thereof, comprising culturing a host cell as provided; and isolating the binding molecule or fragment thereof or antibody or fragment thereof.

In certain embodiments, the polynucleotides comprise the coding sequence for the mature pan-filovirus binding molecule, e.g., a cross-reactive anti-filovirus antibody or antigen-binding fragment thereof, fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) can be used.

Polynucleotide variants are also provided. Polynucleotide variants can contain alterations in the coding regions, non-coding regions, or both. In some embodiments polynucleotide variants contain alterations that produce silent substitutions, additions, or deletions, but do not alter the properties or activities of the encoded polypeptide. In some embodiments, polynucleotide variants can be produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli). Vectors and cells comprising the polynucleotides described herein are also provided.

In some embodiments, a DNA sequence encoding a pan-filovirus binding molecule, e.g., a cross-reactive anti-filovirus antibody or antigen-binding fragment thereof can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest can be inserted into an expression vector and operatively linked to an expression control sequence appropriate for expression of the protein in a desired host. Proper assembly can be confirmed, e.g., by nucleotide sequencing, restriction mapping, and/or expression of a biologically active polypeptide in a suitable host. In order to obtain high expression levels of a transfected gene in a host, the gene can be operatively linked to or associated with transcriptional and translational expression control sequences that are functional in the chosen expression host.

In certain embodiments, recombinant expression vectors are used to amplify and express DNA encoding a pan-filovirus binding molecule, e.g., a cross-reactive anti-filovirus antibody or antigen-binding fragment thereof. Recombinant expression vectors are replicable DNA constructs which have synthetic or cDNA-derived DNA fragments encoding a polypeptide chain of an anti-filovirus antibody or and antigen-binding fragment thereof, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine. This methionine can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

Suitable host cells for expression of a pan-filovirus binding molecule, e.g., a cross-reactive anti-filovirus antibody or antigen-binding fragment thereof include prokaryotes, yeast, insect or higher eukaryotic cells under the control of appropriate promoters. Prokaryotes include gram negative or gram-positive organisms, for example E. coli or bacilli. Higher eukaryotic cells include established cell lines of mammalian origin as described below. Cell-free translation systems could also be employed. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems can also be employed to express a pan-filovirus binding molecule, e.g., a cross-reactive anti-filovirus antibody or antigen-binding fragment thereof. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as ribosome binding sites, a polyadenylation site, splice donor and acceptor sites, and transcriptional termination sequences. Baculovirus systems for production of heterologous proteins in insect cells are reviewed by Luckow and Summers, BioTechnology 6:47 (1988).

A pan-filovirus binding molecule, e.g., a cross-reactive anti-filovirus antibody or antigen-binding fragment thereof produced by a transformed host, can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a pan-filovirus binding molecule, e.g., a cross-reactive anti-filovirus antibody or antigen-binding fragment thereof. Some or all of the foregoing purification steps, in various combinations, can also be employed to provide a homogeneous recombinant protein.

A pan-filovirus binding molecule, e.g., a cross-reactive anti-filovirus antibody or antigen-binding fragment thereof produced in bacterial culture, can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

Treatment Methods Using Pan-Filovirus Binding Molecules

Methods are provided for the use of pan-filovirus binding molecules, e.g., cross-reactive anti-filovirus antibodies or fragments thereof, to treat patients having a disease or condition associated with a filovirus infection, or to prevent, reduce, or manage filovirus-induced virulence in a subject infected with a filovirus.

The following discussion refers to diagnostic methods and methods of treatment of various diseases and disorders with a pan-filovirus binding molecule, e.g., a cross-reactive anti-filovirus antibody or antigen-binding fragment thereof that retains the desired properties of anti-filovirus antibodies provided herein, e.g., capable of specifically binding to and neutralizing filovirus infectivity and/or virulence. In some embodiments, a pan-filovirus binding molecule, e.g., a cross-reactive anti-filovirus antibody or antigen-binding fragment thereof can be a murine, human, or humanized antibody. In some embodiments, the anti-filovirus antibody or antigen-binding fragment thereof comprises a first binding domain that binds to the same epitope as, or competitively inhibits binding of, one or more of murine monoclonal antibodies m2D8, m21D10, m5E4, m16G8, m17C6, m8C4, m4B8, m21B2, or m2E4 as provided herein. In some embodiments, the first binding domain of an anti-filovirus antibody or antigen-binding fragment thereof as provided herein is derived from one or more of murine monoclonal antibodies m2D8, m21D10, m5E4, m16G8, m17C6, m8C4, m4B8, m21B2, or m2E4 as provided herein. In certain embodiments the first binding domain of the derived antibody is an affinity-matured, chimeric, or humanized antibody. In some embodiments a pan-filovirus binding molecule, e.g., a cross-reactive anti-filovirus antibody or antigen-binding fragment thereof further comprises a second binding domain that can target the first binding domain to the endosome of a virus-infected cell.

In one embodiment, treatment includes the application or administration of a pan-filovirus binding molecule, e.g., a cross-reactive anti-filovirus antibody or antigen-binding fragment thereof as provided herein, to a subject or patient, where the subject or patient has been exposed to a filovirus, infected with a filovirus, has a filovirus disease, a symptom of a filovirus disease, or a predisposition toward contracting a filovirus disease. In another embodiment, treatment can also include the application or administration of a pharmaceutical composition comprising a pan-filovirus binding molecule, e.g., a cross-reactive anti-filovirus antibody or antigen-binding fragment thereof as provided herein, to a subject or patient, so as to target the pharmaceutical composition to an environment where the binding molecule can be most effective, e.g., the endosomal region of a virus-infected cell.

In accordance with the methods of the present disclosure, at least one a pan-filovirus binding molecule, e.g., a cross-reactive anti-filovirus antibody or antigen-binding fragment thereof as defined elsewhere herein, can be used to promote a positive therapeutic response. By “positive therapeutic response” is intended any improvement in the disease conditions associated with the activity of the binding molecule, and/or an improvement in the symptoms associated with the disease. Thus, for example, an improvement in the disease can be characterized as a complete response. By “complete response” is intended an absence of clinically detectable disease with normalization of any previously test results. Such a response can in some cases persist, e.g., for at least one month following treatment according to the methods of the disclosure. Alternatively, an improvement in the disease can be categorized as being a partial response.

Pharmaceutical Compositions and Administration Methods

Methods of preparing and administering a pan-filovirus binding molecule, e.g., a cross-reactive anti-filovirus antibody or antigen-binding fragment thereof provided herein, to a subject in need thereof are well known to or are readily determined by those skilled in the art. The route of administration of a pan-filovirus binding molecule, e.g., a cross-reactive anti-filovirus antibody or antigen-binding fragment thereof can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as suitable forms, another example of a form for administration would be a solution for injection, in particular for intravenous or intraarterial injection or drip. In some cases a suitable pharmaceutical composition can comprise a buffer (e.g. acetate, phosphate or citrate buffer), a surfactant (e.g. polysorbate), optionally a stabilizer agent (e.g. human albumin), etc. In other methods compatible with the teachings herein, a pan-filovirus binding molecule, e.g., a cross-reactive anti-filovirus antibody or antigen-binding fragment thereof as provided herein can be delivered directly to a site where the binding molecule can be effective in virus neutralization, e.g., the endosomal region of a filovirus-infected cell.

As discussed herein, a pan-filovirus binding molecule, e.g., a cross-reactive anti-filovirus antibody or antigen-binding fragment thereof provided herein, can be administered in a pharmaceutically effective amount for the in vivo treatment of diseases or disorders associated with filovirus infection. In this regard, it will be appreciated that the disclosed binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of a pan-filovirus binding molecule, e.g., a cross-reactive anti-filovirus antibody or antigen-binding fragment thereof means an amount sufficient to achieve effective binding to a target and to achieve a benefit, e.g., to ameliorate symptoms of a disease or condition or to detect a substance or a cell. Suitable formulations for use in the therapeutic methods disclosed herein can be described in Remington's Pharmaceutical Sciences (Mack Publishing Co.) 16th ed. (1980).

The amount of a pan-filovirus binding molecule, e.g., a cross-reactive anti-filovirus antibody or antigen-binding fragment thereof that can be combined with carrier materials to produce a single dosage form will vary depending upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a pan-filovirus binding molecule, e.g., a cross-reactive anti-filovirus antibody or antigen-binding fragment thereof can be administered to a human or other animal in accordance with the aforementioned methods of treatment in an amount sufficient to produce a therapeutic effect. A pan-filovirus binding molecule, e.g., a cross-reactive anti-filovirus antibody or antigen-binding fragment thereof provided herein can be administered to such human or other animal in a conventional dosage form prepared by combining the antibody or antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

By “therapeutically effective dose or amount” or “effective amount” is intended an amount of a pan-filovirus binding molecule, e.g., a cross-reactive anti-filovirus antibody or antigen-binding fragment thereof, that when administered brings about a positive therapeutic response with respect to treatment of a patient with a disease or condition to be treated.

Therapeutically effective doses of the compositions disclosed herein, for treatment of diseases or disorders associated with filovirus infection, vary depending upon many different factors, including means of administration, target site, physiological state of the patient, whether the patient is human or an animal, other medications administered, and whether treatment is prophylactic or therapeutic. Usually, the patient is a human, but non-human mammals including non-human primates can also be treated. Treatment dosages can be titrated using routine methods known to those of skill in the art to optimize safety and efficacy.

The amount of a pan-filovirus binding molecule, e.g., a cross-reactive anti-filovirus antibody or antigen-binding fragment thereof to be administered can be readily determined by one of ordinary skill in the art without undue experimentation given this disclosure. Factors influencing the mode of administration and the respective amount of a pan-filovirus binding molecule, e.g., a cross-reactive anti-filovirus antibody or antigen-binding fragment thereof include, but are not limited to, the severity of the disease, the history of the disease, and the age, height, weight, health, and physical condition of the individual undergoing therapy. Similarly, the amount of a pan-filovirus binding molecule, e.g., a cross-reactive anti-filovirus antibody or antigen-binding fragment thereof to be administered will be dependent upon the mode of administration and whether the subject will undergo a single dose or multiple doses of this agent.

This disclosure also provides for the use of a pan-filovirus binding molecule, e.g., a cross-reactive anti-filovirus antibody or antigen-binding fragment thereof in the manufacture of a medicament for treating, preventing, or managing a disease or disorder associated with filovirus infection, e.g., hemorrhagic fever.

Kits Comprising Pan-Filovirus Binding Molecules

This disclosure further provides kits that comprise a pan-filovirus binding molecule, e.g., a cross-reactive anti-filovirus antibody or antigen-binding fragment thereof as described herein and that can be used to perform the methods described herein. In certain embodiments, a kit comprises a pan-filovirus binding molecule, e.g., a cross-reactive anti-filovirus antibody or antigen-binding fragment thereof in one or more containers. In some embodiments, the kits contain all of the components necessary and/or sufficient to perform a detection assay, including controls, directions for performing assays, and software for analysis and presentation of results. One skilled in the art will readily recognize that a pan-filovirus binding molecule, e.g., a cross-reactive anti-filovirus antibody or antigen-binding fragment thereof as provided herein can be readily incorporated into one of the established kit formats which are well known in the art.

Immunoassays

A pan-filovirus binding molecule, e.g., a cross-reactive anti-filovirus antibody or antigen-binding fragment thereof can be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety).

The binding activity of a given lot of a pan-filovirus binding molecule, e.g., a cross-reactive anti-filovirus antibody or antigen-binding fragment thereof can be determined according to well-known methods. Those skilled in the art will be able to determine operative and optimal assay conditions for each determination by employing routine experimentation.

Methods and reagents suitable for determination of binding characteristics of a pan-filovirus binding molecule, e.g., a cross-reactive anti-filovirus antibody or antigen-binding fragment thereof are known in the art and/or are commercially available. Equipment and software designed for such kinetic analyses are commercially available (e.g., BIAcore®, BIAevaluation® software, GE Healthcare; KINEXA® Software, Sapidyne Instruments).

### EXAMPLES

### Example 1: Generation of Mouse Monoclonal Antibodies Against Filovirus Glycoproteins

A. Methods

Production and Purification of Filovirus Glycoproteins.

Plasmids for the following glycoprotein (GP) constructs were generated: full ectodomain of the glycoprotein (GPΔTM) for EBOV (Mayinga) and SUDV (Boniace) containing amino acids 1-627 with a hemagglutinin (HA) tag (YPYDVPDYA (SEQ ID NO: 113)) followed by a Factor 10a cleavage site (IEGRGAR (SEQ ID NO: 114)); truncated forms of EBOV GP ectodomains lacking the mucin-like domains (MLD) (GPΔmuc) and consisting of amino acids 1-311 followed by an aspartic acid and linked to amino acids 464-637; and SUDV GPΔmuc consisting of amino acids 1-313 linked to amino acids 474-640 were generated and expressed in 293T cells. GPΔTM proteins were purified from the supernatants using the method described by Lee et al., Nature 454, 177-182 (2008). Briefly, proteins expressed in 293T cells were purified using Q-Sepharose Fast Flow resin. Elutions were pooled and these fractions were passed over a GE S-200 HR column. S-200 fractions containing GPΔTM were pooled and dialyzed against PBS. GPΔmuc protein was purified using Q-Sepharose Fast Flow resin and eluted fractions containing the proteins were pooled and dialyzed against PBS. For insect cell expression of GPΔTM for EBOV, SUDV, RESTV, and BDBV the coding regions for amino acids 1-605 (1-609 for BDBV) followed by a C-terminal 6×His tag were cloned into the baculovirus transfer vector pFastBac (Invitrogen) containing the polyhedrin late promoter and SV40 polyadenylation site. Bacmid DNA was produced by in vivo transposition in E. coli as described (Warfield, K. L., et al. PLoS One 10: e0118881 (2015)). Upon transfection of Sf9 insect cells by the bacmid DNA the recombinant baculoviruses containing the GPΔTM were recovered from supernatants and amplified by another passage in Sf9 cells. The final virus was used to infect Sf9 cells for purification of the proteins from the supernatants three days post infection. After separation of cell debris the supernatants were concentrated 10× by TFF. The concentrate was mixed with 2 mM CaCl₂, 0.25 mM Ni²⁺, 20% Glycerol, 10 mM imidazole, 0.5% Triton X-100 and 1M NaCl (final concentration), then the pH adjusted to 7.2. Ni beads (Ni Sepharose 6 Fast Flow, GE Life Sciences, 17-5318-01) were added at 1 ml per liter of concentrated supernatant and mixed overnight at 4° C. Ni beads were separated from the supernatant by centrifugation, resuspended in PBS+0.2% Tween 20 and packed into a chromatography column. The column was washed with the following buffers: Phosphate buffered saline pH 7.1 (PBS) supplemented with 20% glycerol and 0.2% Tween 20 followed by PBS supplemented with 20% glycerol, 0.2% Tween 20 and 10 mM Imidazole. Protein was eluted with PBS containing 500 mM imidazole. Eluted proteins were dialyzed against PBS supplemented with 10% glycerol containing arginine and glutamic acid, pH 7.4. For production of soluble GP (sGP), the full coding sequence of EBOV sGP including delta peptide followed by a C-terminal Tag was expressed in 293T cells. The supernatants were passed through a Ni column to separate the delta peptide. The flow through was concentrated and used as source of sGP. Purified proteins were analyzed by standard BCA, SDS-PAGE, and Western Blot.

Enzymatic Cleavage of GP and Purification.

Proteolytically cleaved GP ectodomains were produced as described (Hashiguchi, T., et al. Cell 160, 904-912 (2015)). Briefly, Effectene Reagent (Qiagen) was used to transfect S2 cells with pMTpuro plasmids containing a strep-tagged filovirus mucin-deleted GP gene, followed by stable selection of transfected cells with 6 μg/ml puromycin in Insect XPRESS protein free medium (Lonza). Secreted GP ectodomain expression was induced with 0.5 mM CuSO₄ and supernatants harvested after 4 days. Proteins were affinity purified using Streptactin resin (Qiagen). The cleaved “core” ectodomain for MARV (MARV GPcl) was produced by incubating 1 mg Ravn GPΔmuc with 0.01 mg trypsin (Sigma) at 37° C. for 1 hour in TBS pH 7.5, followed by S200 size exclusion (SEC) purification. The cleaved “core” ectodomain of EBOV (EBOV GPcl) was produced by incubating 1 mg EBOV GPΔmuc with 0.02 mg thermolysin (Sigma) overnight at room temperature in TBS buffer plus 1 mM CaCl₂, followed by S200 SEC purification.

Immunization.

Female BALB/c mice (6-8 weeks old) were immunized with a combination of GPΔmuc proteins for EBOV (Mayinga), SUDV (Boniface), and MARV (Musoke) (25 μg each) on study days 0, 14 and 28 (Group 1) or study days 0, 14, 28, 42 and 56 along with 20 μg Sigma Adjuvant System (Sigma) by the intramuscular (IM) route. An intravenous dose of the same antigens without adjuvant was administered on day 35 (Group 1) or day 63 (Group 2). Vaccines were received via intramuscular (IM) or subcutaneous (SQ) route of delivery with monophosphoryl lipid A (MPL) adjuvant. Mice were vaccinated every 2 weeks for 1-2 months and a booster injection was given three days prior to harvest via intravenous (IV) route. Bleeds were performed by tail vein nick under mild physical restraint and without anesthesia and tested using a binding ELISA to determine the antibody response. After the final immunization, the mice were sacrificed following cardiac puncture and blood collection. The experiments involved injecting no more than 100 μl (0.1 ml) material at a time. Following the final immunization the mice will be sacrificed following blood collection by cardiac puncture. Spleens will be harvested for isolation of B cells and fusion for production of hybridoma clones.

Fusion and Screening:

Mice were euthanized three days after the last intravenous boost. Cells were harvested from the spleens and lymph nodes and 1×10⁸ lymphocytes/splenocytes were fused with the 2×10⁷ SP2/0-Ag14 myeloma cells (ATCC), using polyethylene glycol (PEG). Fused cells were incubated overnight in Hybridoma Recovery Medium to allow the fused cells to go through one cell cycle to express the enzyme hypoxanthine-guanine phosphoribosyltransferase (HRPT) that will allow them to survive in selection medium. The day after the fusion, cells were plated in methylcellulose-containing Hybridoma Selection Medium containing hypoxanthine, aminopterin, and thymidine (HAT) in 96-well plates. Plates were left undisturbed for 10-14 days at 37° C., 5% CO₂ incubator. Plates were fed with Hybridoma growth Medium containing HT every few days. Wells with cell growth that covered ≧25% of the well area and medium turning yellow were screened to determine if the wells contained hybridoma clones that produced antibodies against EBOV, SUDV and MARV glycoproteins. Cells from wells identified as positive against two or all three glycoproteins in the screening ELISA, were expanded and re-cloned by limiting dilutions.

Animal immunization studies were approved by the Nobel Life Sciences (Gaithersburg, Md.) Institutional Animal Care and Use Committee (IACUC). The animal facility used is fully accredited by the Association for Assessment and Accreditation of Laboratory Animal Care.

Screening ELISA to Test Hybridoma Supernatants:

Purified recombinant glycoproteins without the transmembrane region (GPΔTM) or mucin-like domain and transmembrane region (GPΔmuc) were immobilized at 100 ng/well on 96-well Nunc MaxiSorp plates (ThermoFisher Scientific) and incubated with dilutions of hybridoma supernatants or serial dilutions of purified mouse monoclonal antibodies. Bound antibodies were detected using an HRP-conjugated anti-mouse secondary antibody (KPL) and 3,3′, 5,5′ tetramethylbenzidine (TMB) substrate (Life Technologies). Supernatants were considered positive if the absorbance value determined at 650 nm was at least two times above background.

ELISA to Test Purified Mouse Monoclonal Antibodies:

Purified GPΔmuc or GPΔTM or cathepsin-cleaved GP were immobilized on 96-well Nunc MaxiSorp plates (ThermoFisher Scientific) and incubated with serial dilutions of purified mouse monoclonal antibodies. Bound antibodies were detected using an HRP-conjugated anti-mouse secondary antibody (KPL) and TMB substrate (Life Technologies). Absorbance values determined at 650 nm were transformed using Softmax® 4 parameter curve-fit (Molecular Devices). Half maximal effective concentration (EC50) values at the inflection point of the curve were determined.

Western Blot:

Purified recombinant glycoproteins GPΔTM or GPΔmuc were heat-denatured and treated with reducing sample buffer before being separated on a gradient gel. Separated protein bands were transferred onto PVDF membranes and probed with the mouse monoclonal antibody of interest and detected with anti-mouse secondary antibody with alkaline phosphatase (AP) conjugate and visualized using AP substrate.

Affinity Measurement:

Mouse monoclonal antibodies were evaluated using a ForteBio OctetRed96 unit. The specific monoclonal antibody was immobilized on the biosensor tip using Fc-capturing biosensors. The immobilized monoclonal antibody biosensor is incubated in PBS to determine a baseline. Various concentrations of antigen are incubated with immobilized monoclonal antibody biosensor to measure association, and then incubated in PBS to monitor dissociation.

B. Antibody Generation

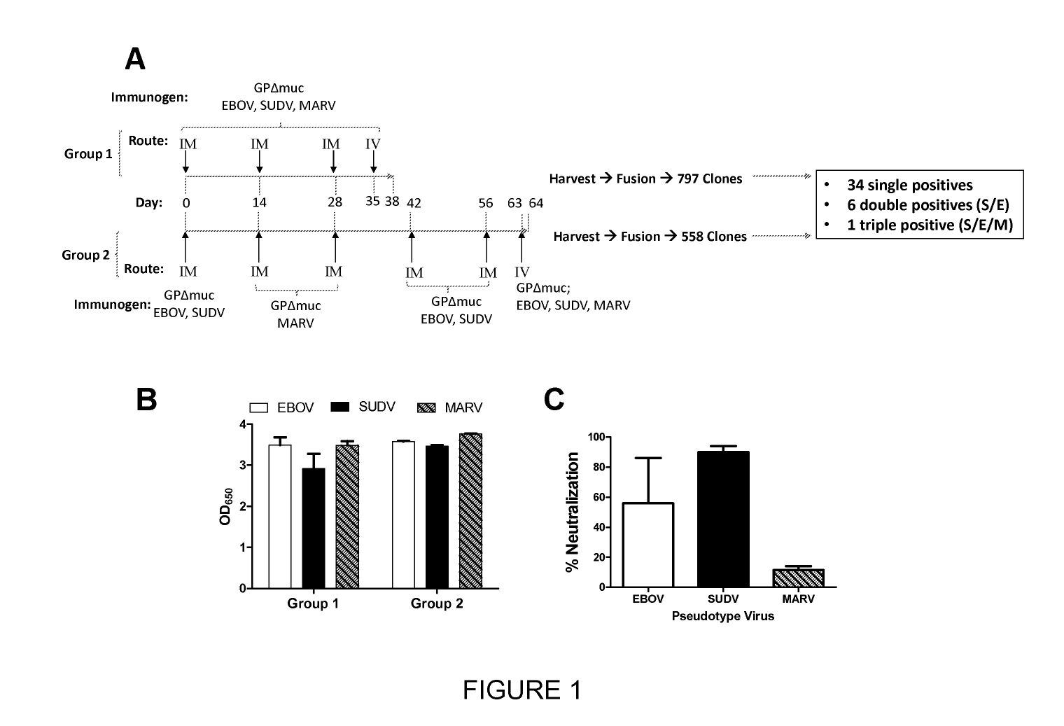

To drive the generation of broadly reactive antibodies BALB/c mice were immunized with a mixture of purified, engineered glycoprotein ectodomains lacking the highly divergent mucin like domain (MLD) (GPΔmuc) for EBOV (Mayinga), SUDV (Boniface), and MARV (Musoke) formulated with Sigma Adjuvant System. Two different immunization strategies were employed as shown in FIG. 1A. Mice were also boosted with the same protein cocktail (without adjuvant) 3 days (Group 1) or one day (Group 2) before euthanasia and harvest of splenocytes followed by fusion and hybridoma development. Total IgG and neutralizing responses against the three filovirus GPs were determined in terminal bleeds (FIGS. 1B and 1C). Splenocytes were harvested and fusion performed to generate hybridomas as described above.

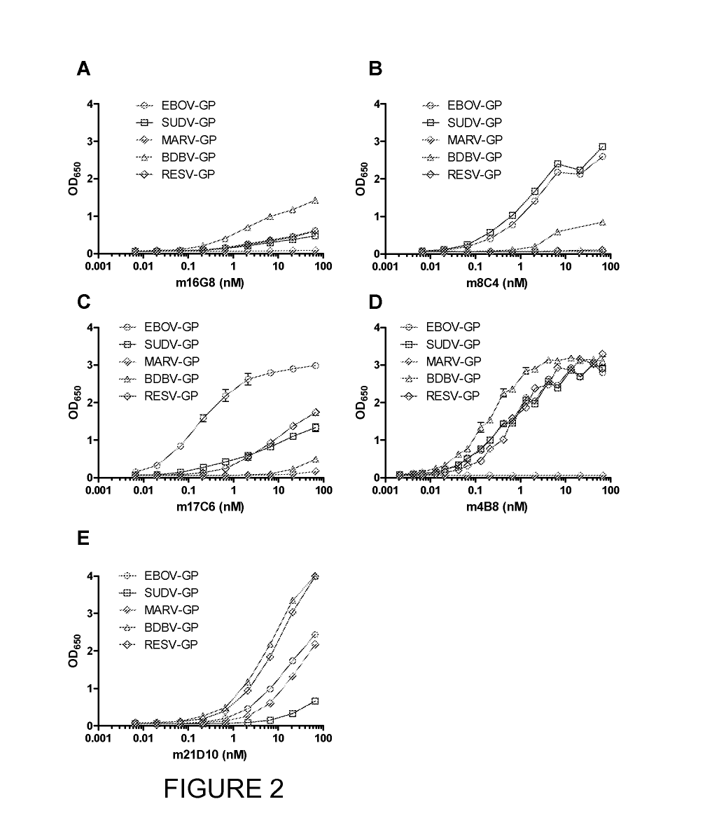

Over 1300 clones were screened initially for binding to the GP ectodomain (GPΔTM) for EBOV, SUDV, and MARV. As expected, the majority of the reactive clones were specific for a single GP species and were excluded from further analysis. A single mAb (m21D10) reactive to all three GP molecules and six clones reactive to both EBOV and SUDV but non-reactive to MARV were identified and grown for further analysis. Upon secondary screening four cross reactive but ebolavirus-specific clones (m16G8, m8C4, m17C6, and m4B8) exhibited greater strength and breadth of binding. These four clones along with m21D10 were further subcloned to ensure clonal purity and the mAbs were produced, purified over protein G chromatography column, and analyzed for binding to full length GP ectodomains from EBOV, SUDV, BDBV, RESTV, and MARV. The mAbs displayed various degree of binding to most species of ebolavirus but none other than m21D10 bound to MARV, consistent with the primary screening results (FIG. 2). The monoclonal m16G8 bound moderately to BDBV but with low affinity to EBOV, SUDV, and RESTV glycoproteins (FIG. 2A). m8C4 showed preferential binding to SUDV and EBOV and to lesser extent to BDBV but failed to bind to RESTV (FIG. 2B). m17C6 displayed strong binding to EBOV but lower level of binding to SUDV and RESTV and very poor binding to BDBV (FIG. 2C). The strongest and most balanced binding was displayed by m4B8 which bound to all four ebolavirus species tested at low nM concentrations (FIG. 2D). The only MARV-reactive clone, m21D10, exhibited the highest binding for BDBV and RESTV followed by MARV, EBOV and low level of binding to SUDV (FIG. 2E), thus representing a pan-filovirus antibody. Subtype-specific ELISA showed that m16G8 is an IgG2a while the other mAbs are IgG1 (data not shown).

C. Reactivity of the mAbs

Nine monoclonal antibodies were identified with reactivity to the glycoprotein of more than one species. Table 2 summarizes the reactivity of these antibodies (by any method, i.e. ELISA, Western blot, or BIACORE) to the glycoproteins from Zaire ebolavirus (EBOV), Sudan ebolavirus (SUDV), Marburg virus (MARV; Musoke isolate), Reston ebolavirus (RESTV), and Bundibugyo ebolavirus (BDBV). Furthermore, the reactivity of two of the mAbs was tested against cathepsin-cleaved EBOV glycoprotein. Three of the mAbs (m2D8, m21D10, and m5E4) showed broad reactivity across ebolavirus species tested as well as Marburg virus, thus referred to here as pan filovirus antibodies. One antibody (m4B8) is a pan-Ebola antibody recognizing all ebolavirus species but not Marburg virus. The remaining mAbs show reactivity to at least two virus species as indicated in Table 2.

TABLE 2
Reactivity of anti-filovirus monoclonal antibodies to glycoproteins
from different species, as well as cathepsin-cleaved EBOV GP

| | | | | | | Cathepsin- |
|---|---|---|---|---|---|---|
| | Reactivity | | | | | cleaved |
| Mab | EBOV | SUDV | MARV | RESTV | BDBV | EBOV |
| m2D8 | + | + | + | + | + | + |
| m21D10 | + | + | + | + | + | + |
| m5E4 | + | + | + | NT | NT | NT |
| m16G8 | + | + | − | + | + | NT |
| m17C6 | + | + | + | − | + | NT |
| m8C4 | + | + | − | − | + | NT |
| m4B8 | + | + | − | + | + | NT |
| m21B2 | − | + | + | NT | NT | NT |
| m2E4 | + | + | − | + | − | NT |

NT = Not Tested

The variable regions of the heavy and light chains of the monoclonal antibodies were sequenced using PCR amplified cDNA fragments reverse transcribed from RNA isolated from the respective hybridoma cells (Table 3).

TABLE 3
Sequence Identifiers

| | Antibody | | | | | | | | |
|---|---|---|---|---|---|---|---|---|---|
| | m2D8 | M21D10 | m5E4 | m16G8 | m17C6 | m8C4 | m4B8 | m21B2 | m2E4 |
| Structure | SEQ ID NO | | | | | | | | |
| DNA en- | 1 | 11 | 21 | 31 | 41 | 51 | 61 | 71 | 81 |
| coding | | | | | | | | | |
| VH | | | | | | | | | |
| region | | | | | | | | | |
| VH | 2 | 12 | 22 | 32 | 42 | 52 | 62 | 72 | 82 |
| entire | | | | | | | | | |
| VH | 3 | 13 | 23 | 33 | 43 | 53 | 63 | 73 | 83 |
| CDR1 | | | | | | | | | |
| VH | 4 | 14 | 24 | 34 | 44 | 54 | 64 | 74 | 84 |
| CDR2 | | | | | | | | | |
| VH | 5 | 15 | 25 | 35 | 45 | 55 | 65 | 75 | 85 |
| CDR3 | | | | | | | | | |
| DNA en- | 6 | 16 | 26 | 36 | 46 | 56 | 66 | 76 | 86 |
| coding | | | | | | | | | |
| VL | | | | | | | | | |
| VL | 7 | 17 | 27 | 37 | 47 | 57 | 67 | 77 | 87 |
| entire | | | | | | | | | |
| VL | 8 | 18 | 28 | 38 | 48 | 58 | 68 | 78 | 88 |
| CDR1 | | | | | | | | | |
| VL | 9 | 19 | 29 | 39 | 49 | 59 | 69 | 79 | 89 |
| CDR2 | | | | | | | | | |
| VL | 10 | 20 | 30 | 40 | 50 | 60 | 70 | 80 | 90 |
| CDR3 | | | | | | | | | |

To determine the relative binding of the mAbs to the glycoproteins from different species semi-quantitative ELISA was performed, in which the binding of the mAbs was examined over a range of mAb concentrations on ELISA plates coated with either GPΔTM or GPΔmuc from different filovirus species. EC50 values, defined as concentration of the antibody leading to 50% maximal binding, were determined. The data are shown in Table 4.

TABLE 4
Relative binding of anti-filovirus monoclonal antibodies to GPΔmuc and
GPΔTM as well as cathepsin-cleaved EBOV GP as determined by ELISA

| | | | | | | | | | ELISA |
|---|---|---|---|---|---|---|---|---|---|
| | | | | | | | | | EC50 |
| | | | | | | ELISA EC50 μg/ml | | | μg/ml |
| | ELISA EC50 μg/ml (GPΔTM) | | | | | (GPΔmuc) | | | (Cleaved |
| Mab | EBOV | SUDV | MARV | RESTV | BDBV | EBOV | SUDV | MARV | EBOV GP) |
| m2D8 | >10 | >10 | 0.0445 | NT | NT | 26.2 | >10 | 0.0573 | 0.72 |
| m21D10 | 1.22 | >10 | 4.22 | 1.84 | 1.07 | 2.22 | 1.33 | 1.8 | 0.12 |
| m16G8 | >10 | >10 | ND | >10 | 0.5 | 1.23 | 0.22 | ND | NT |
| m17C6 | 0.04 | 2 | ND | 1.56 | >10 | 0.03 | 0.03 | ND | NT |
| m8C4 | 0.26 | 0.15 | ND | ND | >10 | 0.21 | 0.06 | ND | NT |
| m4B8 | 0.088 | 0.088 | ND | 0.157 | 0.033 | 0.69 | 0.095 | ND | NT |
| m21B2 | ND | >10 | 0.0688 | NT | NT | >10 | >10 | 0.0307 | NT |
| E4 | >10 | >10 | ND | >10 | 0.69 | 1.27 | 2.31 | ND | NT |

ND = Not Detected
NT = Not Tested
The values represent the concentration of the antibody required for 50% maximum binding (EC₅₀)

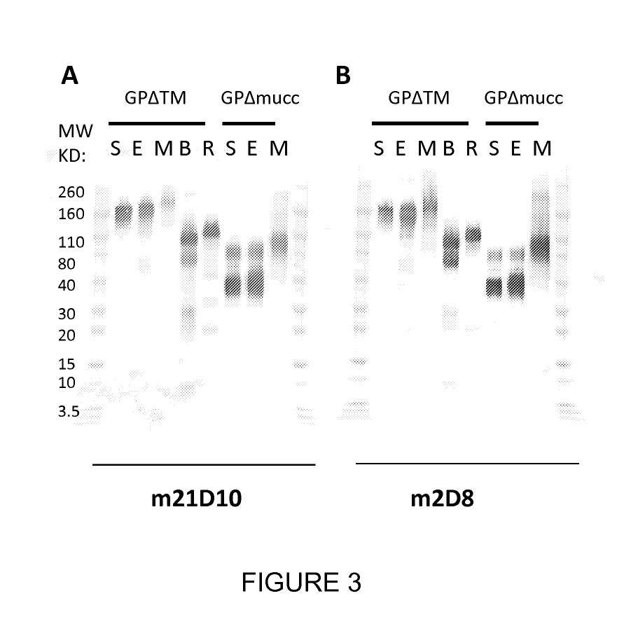

Reactivity of the mAbs was also tested in Western blot assay using GPΔTM and GPΔmuc proteins from various species. As shown in Table 5, the panfilovirus mAbs m2D8 and m21D10 showed broad reactivity suggesting that these two antibodies recognize a shared linear epitope among filovirus species. In contrast other mAbs except for m21B2 did not detect the denatured glycoproteins suggesting that they are specific to conformational epitopes. FIG. 3 shows representative Western blot data with mAbs m21D10 (panel A) and m2D8 (panel B) demonstrating reactivity to GP from all filovirus species tested (EBOV, SUDV, MARV, Reston, and Bundibugyo). m21D10 reacted with chemically denatured antigen (data not shown) and recognized GP from EBOV, SUDV, BDBV, RESTV, and MARV in Western blot analysis (FIG. 3A) indicating that it binds to a continuous epitope. Four pan-ebolavirus mAbs (m16G8, m8C4, m17C6, and m4B8) failed to detect GP under denaturing conditions (data not shown), suggesting that they react with discontinuous epitopes.

TABLE 5
Reactivity of anti-filovirus monoclonal antibodies to glycoproteins
from different species as determined by Western blot analysis

| | | | | | | Western Blot Reactivity to | | |
|---|---|---|---|---|---|---|---|---|
| | Western Blot Reactivity to GPΔTM | | | | | GPΔmuc | | |
| Mab | EBOV | SUDV | MARV | RESTV | BDBV | EBOV | SUDV | MARV |
| m2D8 | + | + | + | + | + | + | + | + |
| m21D10 | + | + | + | + | + | + | + | + |
| m5E4 | ND | ND | ND | NT | NT | ND | ND | ND |
| m16G8 | ND | ND | ND | NT | NT | ND | ND | ND |
| m17C6 | ND | ND | ND | NT | NT | ND | ND | ND |
| m8C4 | ND | ND | ND | NT | NT | ND | ND | ND |
| m4B8 | ND | ND | ND | NT | NT | ND | ND | ND |
| m21B2 | − | − | + | NT | NT | − | − | + |
| m2E4 | ND | ND | ND | NT | NT | ND | ND | ND |

ND = Not Detected
NT = Not Tested

C. Affinity Measurements

Affinity of three of the panfilovirus mAbs to GPΔmuc proteins from EBOV, SUDV, and MARV was determined using ForteBio OctetRed96. Table 6 shows the results of this study.

TABLE 6
Dissociation constant of pan-filovirus antibodies
to GPΔmuc of different species

| | KD (Molar) | | |
|---|---|---|---|
| mAb | EBOV | SUDV | MARV |
| m17C6 | 1.1 × 10−8 | 2.8 × 10−9 | 3.2 × 10−8 |
| m21D10 | 1.8 × 10−8 | 2.7 × 10−9 | 4.1 × 10−8 |
| m5E4 | 1.2 × 10−8 | 2.8 × 10−9 | 3.0 × 10−8 |

In certain aspects, affinity maturation of the panfilovirus antibodies, e.g., by amino acid substitutions the complementarity determining regions (CDRs) or framework regions, can be performed to modulate binding affinities and also to modify the relative binding to different species and achieve a more balanced binding profile.

In certain aspects the panfilovirus antibodies can be expressed as chimeric antibodies (e.g., the murine VH and VL variable regions are spliced to human constant regions), or as humanized antibodies, in which the murine CDRs or variants thereof are grafted into human framework regions, all as described elsewhere herein.

### Example 2: Epitope Mapping

A. Methods

Competition ELISAs:

Purified C-terminal His-tagged EBOV GPΔTM was immobilized at 100 ng/well on 96-well Nickle coated plates (Promega) at 4° C. overnight. The next day, plates were washed, blocked for 1 hour, and set of two mAbs were then added and allowed to bind for 1 hour at room temperature. To detect competition between mouse and humanized mAbs, 1 μg/ml mouse mAb (4 μg/ml for m8C4) was mixed with a 10-fold excess of humanized mAb and detected using 1:2000 dilution of goat anti-mouse-HRP (Bio-Rad). To detect competition between two mouse mAbs, m8C4 was biotinylated with EZ-Link NHS-biotin according to the manufacture's protocol (Life Technologies) and detected with a 1:4000 dilution of anti-streptavidin-HRP (KPL). KZ52 competition with humanized mAbs also required biotinylation and detection with anti-streptavidin-HRP. After 1 hour, TMB substrate (Life Technologies) was added for 30 min at room temperature. Absorbance values determined at 650 nm on a VersaMax plate reader. Percent competition values were calculated from mAb binding in the presence of an irrelevant mAb control and rounded to the nearest whole number.

Epitope Mapping:

ELISA was performed to confirm that a particular mouse monoclonal antibody would recognize a linear (continuous) epitope on GPΔmuc protein. One concentration of mouse monoclonal antibody was selected and incubated with overlapping peptides corresponding to the primary sequence of SUDV, EBOV and MARV glycoproteins (sources: GenScript, ProImmune, and Mimotopes). The peptides recognized by the antibody, bound to the antibody during pre-incubation, would prevent the antibody from binding to rGPΔmuc-coated wells, thus lowering the absorbance values compared to that of the antibody only control.

B. Results

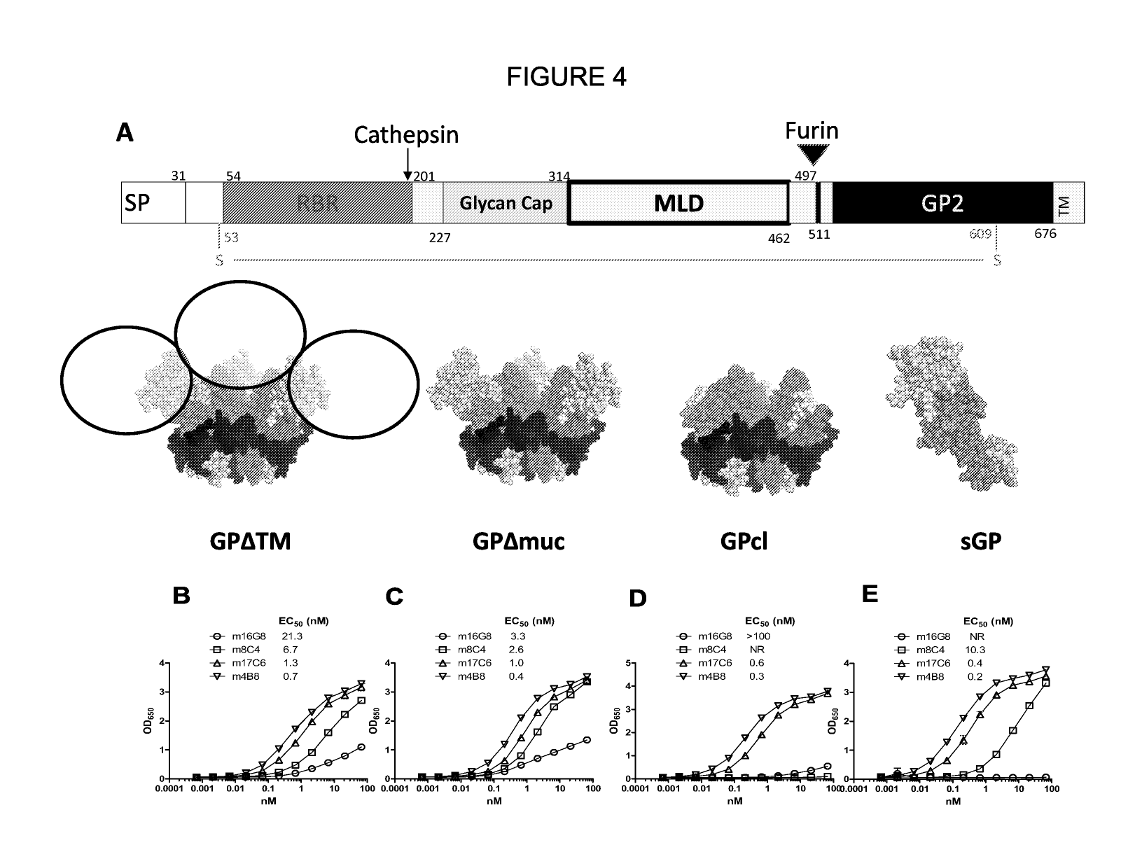

To determine the general binding regions of the conformational antibodies, we compared the binding of the mAbs to several EBOV GP constructs containing various functional domains of the protein. EBOV GP consists of the disulfide bound GP1 and GP2 which are responsible for receptor binding and membrane fusion respectively (FIG. 4A). GP1 forms a chalice consisting of the receptor binding region (RBR) and the glycan cap positioned at the rim of the chalice as well as a C-terminal, highly glycosylated and disordered mucin-like domain (MLD) (FIG. 4A) (Lee, J. E., et al., Nature 454, 177-182 (2008)). GP2 wraps around GP1, and along with the N-terminus of GP1, forms the base of the chalice. Unedited EBOV GP gene encodes for soluble GP (sGP), which consists of amino acids 31-295 followed by a unique C-terminal tail (Sanchez, A., et al., Proc Natl Acad Sci USA 93, 3602-3607 (1996); Volchkov, V. E., et al., Proc Natl Acad Sci USA 95, 5762-5767 (1998).). sGP includes the glycan cap but lacks the MLD and GP2. During viral entry, GP undergoes cleavage by cathepsins at the N-terminus of glycan cap to generate cleaved GP (GPcl), representing truncated GP1 lacking both glycan cap and MLD, that remains associated with GP2. We used GPΔTM, GPΔmuc, GPcl, and sGP, to systematically explore the binding region of the four pan-ebolavirus mAbs. As shown in FIGS. 4B-E, m4B8 and m17C6 bound to all four GP forms indicating that the respective conformational epitopes are in GP1 within the amino acids 31-200. In contrast, m16G8 failed to bind to sGP (FIG. 4E), and since its binding was not dependent on MLD (compare FIGS. 4B-C) its binding domain requires residues in GP2. Due to reduced binding of m16G8 to GPcl (FIG. 4D), it is possible that the epitope for m16G8 in addition comprises residues in GP1, where the binding is affected by cathepsin cleavage. Alternatively, the additional contact points can be within the glycan cap. The mAb m8C4 bound to all GP forms except for GPcl (FIG. 4D) clearly indicating that its epitope lies within the glycan cap.

The epitopes for two monoclonal antibodies m21D10 and m2D8 were identified using a competition assay with overlapping peptides. Prescreening of a panel of overlapping peptides from SUDV GP with these two antibodies narrowed down the binding epitope to a specific area within the receptor-binding region. Based on this preliminary test corresponding peptides from other Zaire and Musoke strains were generated and tested for binding to these antibodies.

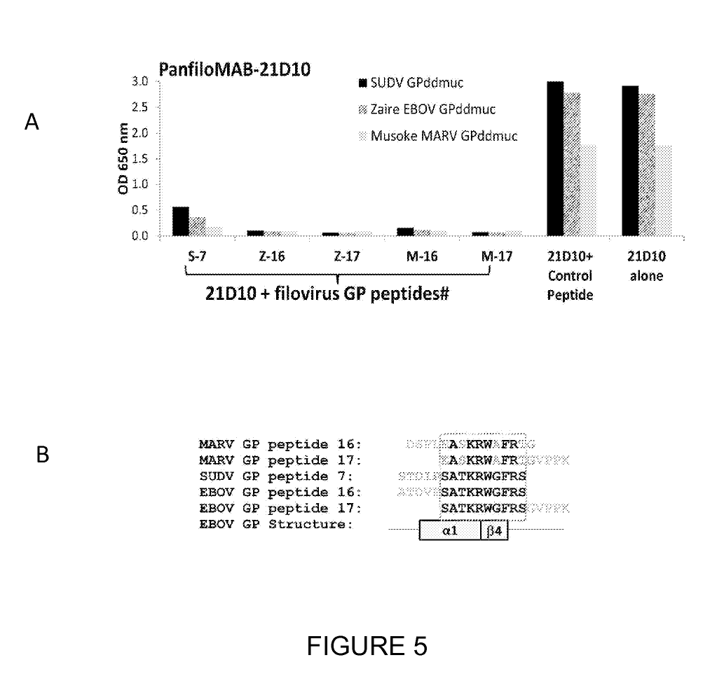

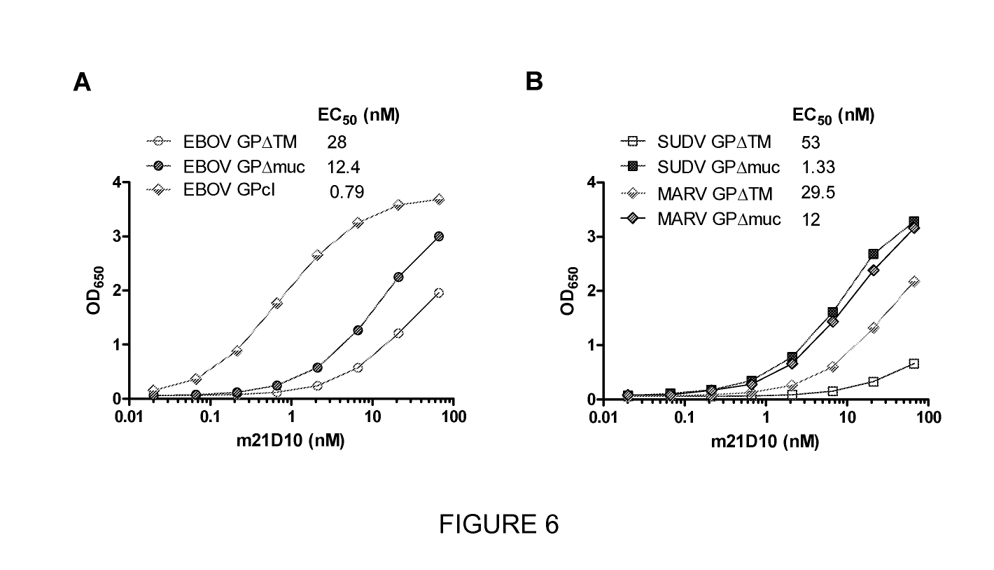

To define the linear epitope of m21D10 we used a library of 15-residue overlapping peptides spanning the entire GP molecules from EBOV, SUDV, and MARV to search for peptides that specifically compete with the binding of the antibody to GP. As shown in FIG. 5A, preincubation of the mAb m21D10 with SUDV GP peptide #7 (corresponding to SUDV GP amino acids 76-90: STDIPSATKRWGFRS (SEQ ID NO: 96)), EBOV Peptides 16 and (overlapping peptides encompassing the EBOV GP amino acids 76-95: ATDVPSATKRWGFRSGVPPK (SEQ ID Nos 97 and 98)), as well as MARV peptides 16 and 17 (overlapping peptides encompassing the MARV GP amino acids 61-79: DSPLEASKRWAFRTGVPPK (SEQ ID Nos 94 and 95)) reduced the binding of m21D10 to all three glycoproteins. In contrast an irrelevant control peptide did not show any competition. This competition assay revealed that m21D10 bound to the residues 81-90 of EBOV and SUDV, as well as positions 65-74 of MARV GP. The alignment of these sequences (FIG. 5B) shows that the consensus sequence of S/E-A-S/T-K-R-W-A/G-F-R-T/S (SEQ ID NO: 109) represents the core binding epitope of m21D10. In three major species of filoviruses, this sequence is located within the receptor binding region (Kuhn, et al., 2006, J Biol Chem, 281 (23):15951-15958) and corresponds to the helix al and strand β4 in the Zaire EBOV GP structure (Lee, et al., 2008, Nature, 454 (7201):177-182). This epitope appears to be heavily concealed on the surface of GP, as removal of the MLD significantly enhanced the binding of m21D10 to EBOV, SUDV, and MARV GP (FIGS. 6A and 6B). The binding of m21D10 to GPcl was significantly higher compared to GPΔmuc indicating that the glycan cap also restricts access to this site (FIG. 6A).

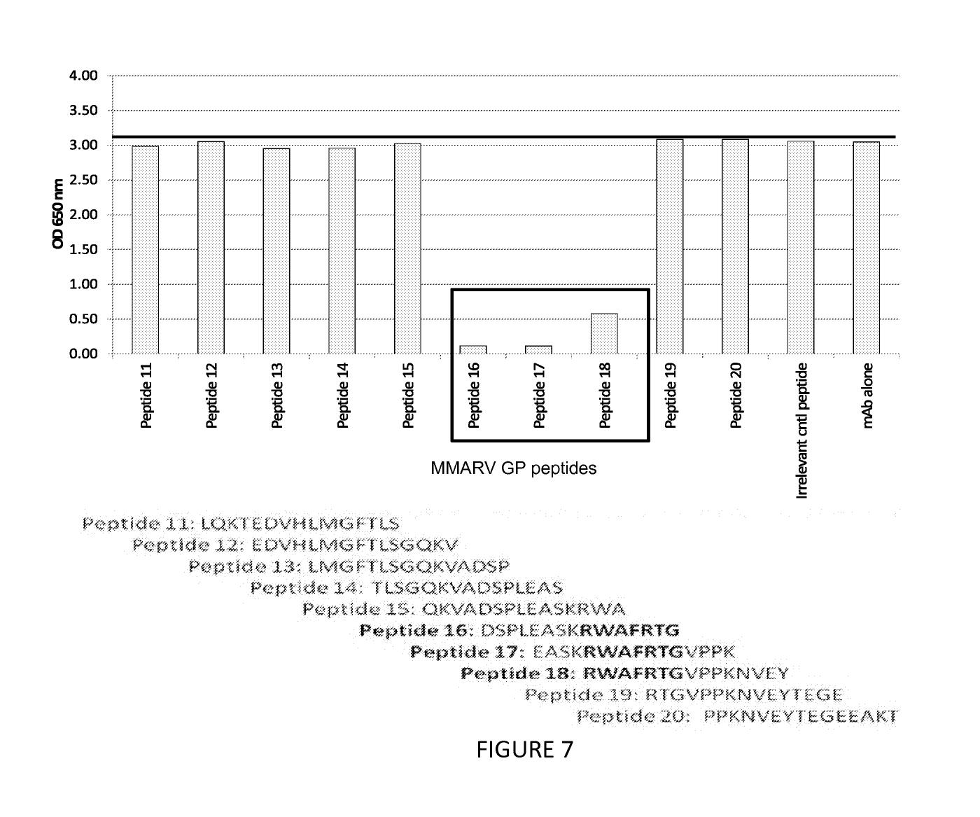

Similarly the binding epitope of pan-filovirus mAb m2D8 was determined using a panel of overlapping peptides. ELISA plates were coated with MARV Musoke GPΔmuc protein. m2D8 mAb at a concentration of 10 μg/ml was incubated with 8 μg/ml of specific peptides denoted below the graph, an irrelevant peptide, or PBS alone. The mixtures were added to the coated plates, incubated for an hour, washed, and bound antibody was determined using a secondary antibody against mouse IgG and detected by TMB substrate. As shown in FIG. 7, the MARV Musoke GP peptides 16, 17, and 18 (SEQ ID Nos 104, 105, and 106) were able to effectively compete with binding of m2D8 to MARV GPΔmuc protein. The consensus core binding sequence derived from these peptides corresponds to MARV GP amino acids 69-75 with sequence of R-W-A-F-R-T-G (SEQ ID NO: 111). This epitope is overlapping with m21D10 epitope with a slight shift towards the C terminus. The core sequence has two amino acid differences to corresponding region in Zaire and Sudan GP (R-W-G-F-R-S-G (SEQ ID NO: 112)). The consensus sequence for the m2D8 epitope is R-W-A/G-F-R-T/S-G (SEQ ID NO: 110).

Using competition ELISA the pan-ebolavirus mAbs were tested for possible epitope overlaps with each other and previously described anti-EBOV mAbs KZ52 (Maruyama, T., et al., J Virol 73, 6024-6030 (1999)) and 13C6 (Olinger, G. G., Jr., et al., Proc Natl Acad Sci USA 109, 18030-18035 (2012); Wilson, J. A., et al., Science 287, 1664-1666. (2000)) (Table 7).

TABLE 7
Percent binding competition between pan-ebolavirus
mAbs, and previously described mAbs KZ52, and
13C6, determined by competition ELISA.*

| | Detec- | | | | | | |
|---|---|---|---|---|---|---|---|
| | tion | | | | | | |
| | anti- | Competing Antibody | | | | | |
| Antigen | body | m8C4 | m16G8 | h17C6 | h4B8 | KZ52 | c13C6 |
| EBOV GP | m8C4- | | 1 | 11 | 8 | 58 | 59 |
| | Biotin | | | | | | |
| | m16G8 | 1 | | 10 | 13 | 3 | 11 |
| | m17C6 | 11 | 10 | | 66 | 6 | 14 |
| | m4B8 | 8 | 13 | 66 | | 2 | 6 |
| | KZ52- | 5 | 6 | 1 | 6 | | 3 |
| | Biotin | | | | | | |
| | c13C6 | 59 | 11 | 14 | 6 | 3 | |
| SUDV GP | m8C4- | | 6 | 5 | 3 | 8 | 0 |
| | Biotin | | | | | | |

*To enable detection of the antibodies in presence of each other some antibodies were biotinylated (m8C4 and KZ52), while some were used as humanized (h4B8, h17C6) or mouse-human chimeric forms (c13C6).

KZ52 binds at the base of the GP chalice (Lee, J. E., et al., Nature 454, 177-182 (2008).) to an epitope shared by ZMapp components 2G4 and 4G7 (Murin, C. D., et al. Proc Natl Acad Sci USA 111, 17182-17187 (2014)), while 13C6, another component of ZMapp (Qiu, X., et al., Nature, 14(7520), 47-53 (2014)), is a glycan cap binder (Murin, C. D., et al., Proc Natl Acad Sci USA 111, 17182-17187 (2014)). m16G8 did not show significant competition with any of the four pan-ebolavirus mAbs, KZ52, or 13C6 and thus recognizes a unique epitope with little steric interference by the other mAbs tested. The glycan cap binder m8C4 showed no competition with the other three pan-ebolavirus mAbs but partially competed with 13C6 for binding to EBOV GP. However, 13C6 did not compete with m8C4 for binding to SUDV GP. Accordingly, m8C4 and 13C6 share contact sites on EBOV glycan cap but not on SUDV and therefore the epitopes for these two mAbs are related but not identical. 13C6 did not compete with any other antibody tested. Partial competition was observed between m17C6 and h4B8, consistent with overlapping conformational epitopes. KZ52 also partially displaced m8C4 but m8C4 was unable to displace KZ52. KZ52 did not show competition with m16G8, m17C6, or m4B8.

C. Discussion

Previous results indicated that the most effective EBOV antibodies target the glycan cap (13C6) and the base of the trimeric GP chalice (4G7, 2G4, and KZ52) (Qiu, X., et al., Nature [[need full cite]] (2014); Murin, C. D., et al., Proc Natl Acad Sci USA 111, 17182-17187 (2014)). Since the KZ52 epitope appears to be strictly species-restricted, and none of the pan-ebolavirus mAbs provided herein bind the KZ52-type base epitope, this disclosure provides new, more broadly cross-reactive epitopes that can be exploited for development of broadly protective antibodies. On the other hand, one of our mAbs, m8C4, bound to a conformational epitope within the glycan cap. Although 13C6 partially competed with binding of m8C4 to EBOV GP, it showed no competition with m8C4 binding to SUDV (Table 7). This indicates that m8C4 binds to a novel glycan cap epitope that is related but not identical to 13C6 epitope. Moreover, m8C4 potently neutralized both EBOV and SUDV in the absence of complement in both pseudotyped and live virus assays (see Example 3, below).

Gross epitope mapping was consistent with the conclusion that m16G8 bound to GP2, since this mAb bound only to the forms of GP that retained GP2 while it failed to bind to sGP. However, m16G8 binding to GPcl was much lower than to GPΔmuc or GPΔTM (FIG. 4), suggesting, e.g., that m16G8 makes additional contacts with specific residues that are lost after proteolysis within the cathepsin cleavage loop (residues 190-213), a disordered loop that traverses over GP2 in the trimeric structure connecting the base to glycan cap (Lee, J. E., et al. Nature 454, 177-182 (2008).). The binding of m16G8 was not displaced by an excess of KZ52, indicating that m16G8 engages a distinct epitope from KZ52.

Cross-reactive mAb, m4B8 binds to a conformational epitope within the first ˜170 amino acids of mature GP1. While the details of this epitope remain to be defined by crystallographic analysis, it is a novel epitope because m4B8 did not compete with any known antibodies tested in our study. While m4B8 was cross protective in challenge studies (see Example 4, below), m4B8 did not show neutralizing activity (see Example 3, below). While not wishing to be bound by theory, possible reasons for this result include, without limitation, the involvement of Fc-dependent effector mechanisms, and/or that the in vitro neutralization assays is not be sensitive enough to detect modest neutralizing activities. For example, in plaque reduction assays we did observe low levels of neutralization by m4B8 (data not shown).

The RBR shows high degree of homology among filoviruses, and yet only a single mAb (m21D10) emerged from our screen that bound to RBR (FIGS. 5 and 6). This antibody bound poorly to full GP ectodomain or virus-like particles but the binding was enhanced exponentially upon proteolysis at the cathepsin cleavage site suggesting that access is restricted by MLD and glycan cap. Recently, Flyak et al described several related monoclonal antibodies isolated from a human survivor of MARV infection with various degrees of reactivity to EBOV GP (Flyak, A. I., et al., Cell 160, 893-903 (2015)). Co-crustal structure of one of these mAbs (MR78) showed that it bound to the putative receptor binding site of MARV GP (Hashiguchi, T., et al., Cell 160, 904-912 (2015)). The linear epitope for m21D10 is distinct from the MR78 epitope but does share several contact residues (Hashiguchi, T., et al., Cell 160, 904-912 (2015)).

### Example 3: Virus Neutralization Assays

A. Methods

Neutralization Assays with VSV-GP_{SUDV} and VSV-GP_{EBOV}:

The pan-ebolavirus mAbs were tested for neutralization of EBOV and SUDV using GFP-expressing VSV pseudotyped with EBOV or SUDV GP according to the methods reported in Chandran, K., et al., Science 308, 1643-1645 (2005), using vesicular stomatitis virus pseudotyped to display the GP from either SUDV or EBOV in place of its native G glycoprotein (VSV-GP_{SUDV} or VSV-GP_{EBOV}, respectively). The viral genome encodes an enhanced green fluorescent protein (eGFP) so that infection is scored by counting fluorescent cells after infection. Briefly, the virus-containing supernatants were harvested and concentrated by pelleting through a 10% sucrose cushion. Virus stocks were titered by infecting African Green Monkey kidney (Vero) cells with serial dilutions and counting eGFP-positive cells by fluorescence microscopy. VSV-GP was used to infect Vero cells at approximate multiplicities of infection of 0.1 to 1.0 in Dulbecco's modified Eagle medium (DMEM) containing 2% fetal bovine serum (FBS; Thermo Scientific, Waltham, Mass.), such that 20-200 cells were infected per well. Vero cell monolayers consisting of ˜7.5×10⁴ cells/well in a 48 well plate were incubated for 14-16 hours with pseudotyped virus that had been pre-incubated with dilutions of the IgG. Infection was scored by manually counting eGFP-positive cells under a fluorescence microscope, 14-16 hours after initial exposure.

Neutralization of Live Ebola and Sudan Viruses:

Antibodies were diluted in PBS at 2× the desired final concentrations, mixed with equal volume of live virus (EBOV or SUDV), and the mixture was incubated at 37° C. for 1 hour before adding to Vero cells in 96 well plates. The cells were incubated with mAb/virus inoculum (MOI ˜1) for 1 hour at 37° C. and washed with PBS. Growth media alone without antibody was added to all wells. Cells were fixed at 48 hours post infection and infected cells were determined by IFA using virus specific mAbs and fluorescently labeled secondary antibodies. Percent of infected cells were determined using an Operetta and Harmony software. Data is expressed as the percent of inhibition relative to vehicle control treated cells for both EBOV and SUDV.

B. Results

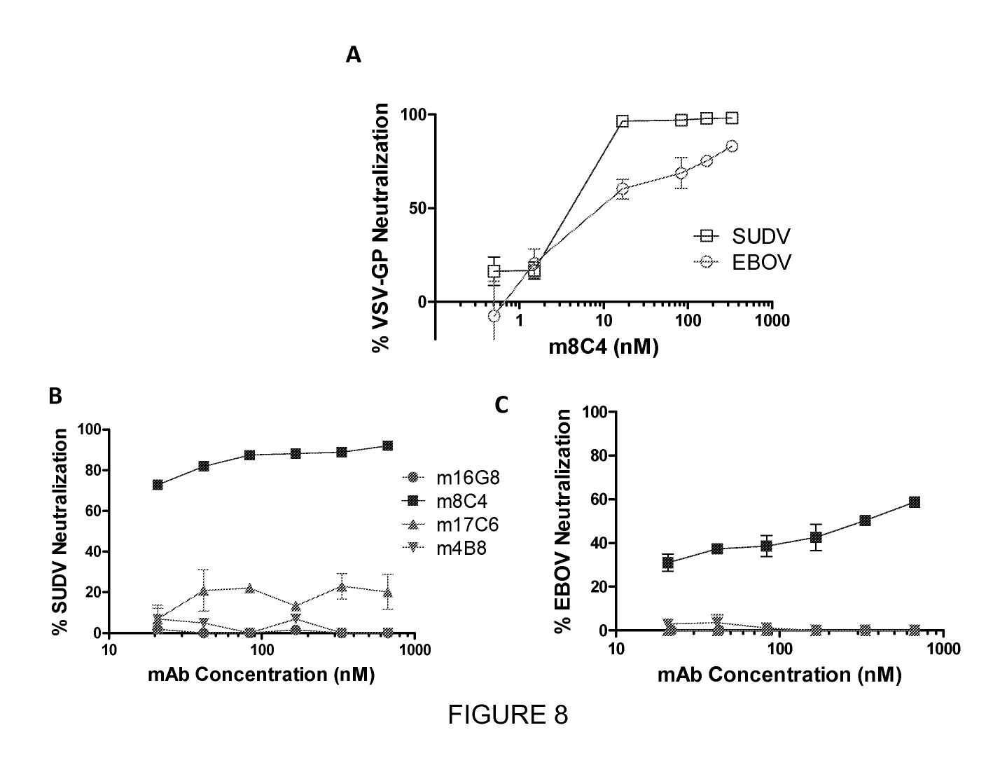

Out of the mAbs tested only m8C4 showed appreciable neutralizing activity in the pseudotyped VSV system. As shown in FIG. 8A, m8C4 effectively inhibited both EBOV GP-VSV and SUDV GP-VSV with 50% inhibitory concentration (IC₅₀) values about 10 and 5 nM respectively. The neutralizing activity of the mAbs was then tested on live virus using a high content imaging-based assay. Consistent with the pseudotype assay, m8C4 effectively neutralized both SUDV (FIG. 8B) and EBOV (FIG. 8C). Low level of neutralization was also observed for m17C6 (FIG. 8C). Neutralization potential of m21D10 was also tested against EBOV, SUDV, and MARV using VSV pseudotype system but this antibody showed no neutralizing activity at up to 66 nM (100 μg/ml) (data not shown).

### Example 4: Testing of Pan-Ebolavirus mAbs in Mouse Models

A. Methods

Murine EBOV Model:

The lethal mouse-adapted EBOV mouse model was developed at USAMRIID, using adult mice by serial passages of EBOV (Zaire) in progressively older suckling mice (Bray, M., et al., J Infect Dis 178(5), 651-61 (1998); Erratum in J Infect Dis 178(5), 1553 (1998)). This model has been thoroughly validated (Gibbs, T. R et al., M. P J Comp Pathol 125, 233-242 (2001)). Female BALB/c mice, aged 6-8 weeks, were purchased from Charles River Laboratory. Upon arrival, mice were housed in micro-isolator cages and provided chow and water ad libitum. On Day 0, mice were transferred to a Biosafety Level 4 containment area and challenged intraperitoneally with a target of 1000 PFU of mouse-adapted EBOV. In two independent experiments groups of mice (n=5, or 10) were treated intraperitoneally with a range of antibody doses, as indicated. Control mice were simultaneously challenged but only given phosphate-buffered saline (PBS) at corresponding treatment intervals. Mice were weighted as groups and monitored daily for 28 days post infection. Where experimental conditions in independent experiments were the same the data for those groups were pooled for graphic presentation in figures and statistical analysis.

Murine SUDV Model:

The SUDV mouse model was developed at USAMRIID utilizing the IFN-α/βR−/− mouse model (Brannan, J. M. et al., J Infect Dis 2015 May 4. pii: jiv215). IFN-α/βR−/− mice (B6.129S2-Ifnar1tm1Agt/Mmjax), aged 8-10 weeks, on the C57BL/6 background were purchased from Jackson Laboratories (Bar Harbor, Me.) and used for all SUDV challenge experiments. Upon arrival, mice were housed in micro-isolator cages and provided chow and water ad libitum. On Day 0, mice were transferred to a Biosafety Level 4 containment area and challenged intraperitoneally with a target of 1000 PFU SUDV-Boniface virus. Groups of mice, n=10 (5 male, 5 female) were treated intraperitoneally with a range of doses of each antibody, as indicated. Control mice were simultaneously challenged but only given phosphate-buffered saline (PBS) at corresponding treatment intervals. Mice were weighted as groups and monitored daily for 28 days post infection.

Animal research was conducted under a protocol approved by the US Army Medical Research Institute of Infectious Diseases (USAMRIID) Institutional Animal Care and Use Committee (IACUC) in compliance with the Animal Welfare Act and other federal statutes and regulations relating to animals and experiments involving animals. The USAMRIID facility is fully accredited by the Association for the Assessment and Accreditation of Laboratory Animal Care International and adheres to the principles stated in the Guide for the Care and Use of Laboratory Animals. Challenge studies were conducted under maximum containment in an animal biosafety level 4 facility. Animal studies were blinded to the personnel performing the work. Animals were not specifically allocated or randomized into groups. The number of mice to be used in these studies was selected to measure and determine differences in levels of protection elicited by the different mAb treatments. Experience with the use of various analyses for determining the probability of differences between control and experimental mouse groups indicates the need for 5-10 mice per group. For lethality studies, statistical difference between 0% in the control and ≧30% in the treated groups can be demonstrated using n=10/group with >90% power using a two-sided alpha level of 0.05. In the cases where large numbers of antibodies were tested in preliminary studies, five mice per group were tested and then results confirmed with the larger group of 5 or 10 mice. In these cases, the results of the combination of both studies are shown. To demonstrate statistical differences in the treatment groups, data were analyzed using GraphPad Software, version 6 (La Jolla, Calif.) using the Mantel-Cox log-rank test and confirmed using the Gehan-Breslow-Wilcoxon test.

B. Challenge with Mouse-Adapted EBOV

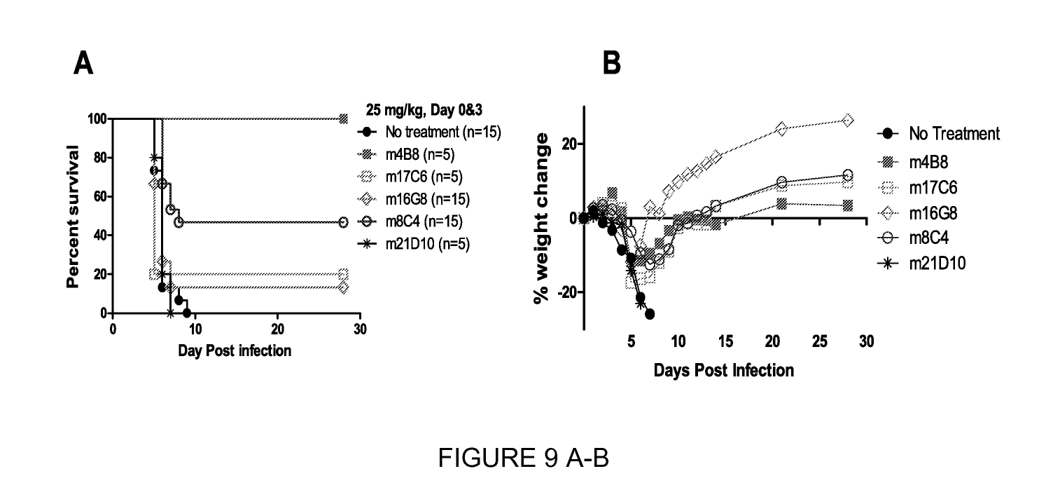

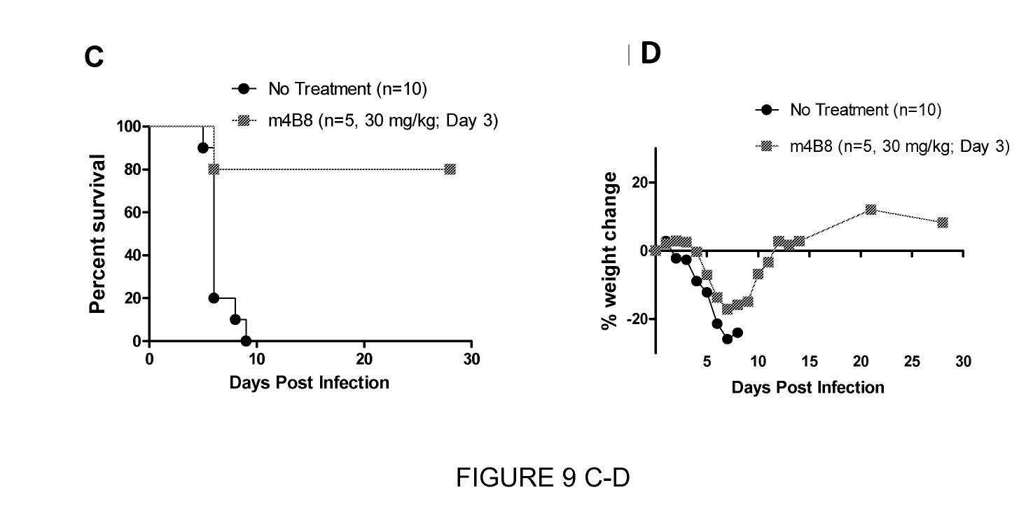

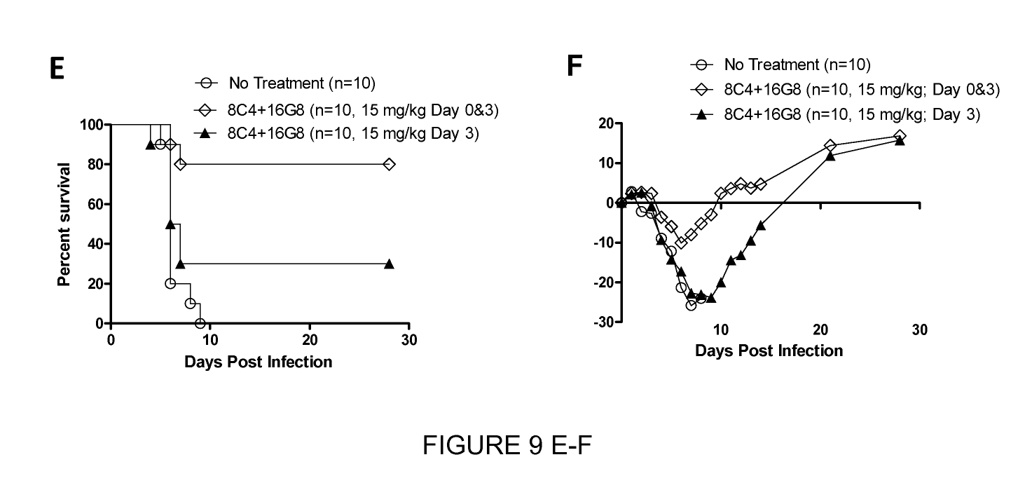

The efficacy of the mAbs was tested in BALB/c using mouse-adapted EBOV (MA-EBOV) (Bray, M. et al., J Infect Dis 178, 651-661 (1998)). Groups of mice were challenged with 1000 plaque-forming units (PFU) of MA-EBOV followed by two intraperitoneal injections of antibody (25 mg/kg) at 2 hours and three days post infection. As shown in FIG. 9A, using this regimen all mice treated with m4B8 and 7 out of 15 mice receiving m8C4 survived the lethal challenge, while m17C6 and m16G8 provided only 20% and 13% protection respectively. In contrast, all 15 controlled mice as well as mice treated with m21D10 succumbed to infection within 5-9 days. Control mice lost more than 25% weight before dying, while all treated animals except for m21D10 group showed significantly lower weight loss (FIG. 9B). Given the strong protection afforded by two injections of m4B8, we examined if delayed treatment with this antibody would provide protection. When treatment with m4B8 was delayed until 3 days after infection 80% of the mice survived the challenge (FIG. 9C) with less than 18% average weight loss (FIG. 9D).

We then tested a combination cocktail of m8C4 (a glycan cap antibody) and m16G8 (an antibody that likely binds GP2). When mice were treated with a cocktail of the two antibodies at 15 mg/kg 80% of the animals survived the challenge (FIG. 9E), in contrast to 47% and 13% survival for m8C4 and m16G8, respectively, when administered individually at a higher dose of 25 mg/kg (FIG. 9A). Delayed treatment (single dose of 15 mg/kg on day 3) with combination of m8C4 and m16G8 also led to 30% survival (FIG. 9E). Mice treated twice with m8C4+m16G8 lost less than 10% weight while the average weight loss in controls was more than 25% (FIG. 9F).

C. Challenge in a SUDV Infection Model

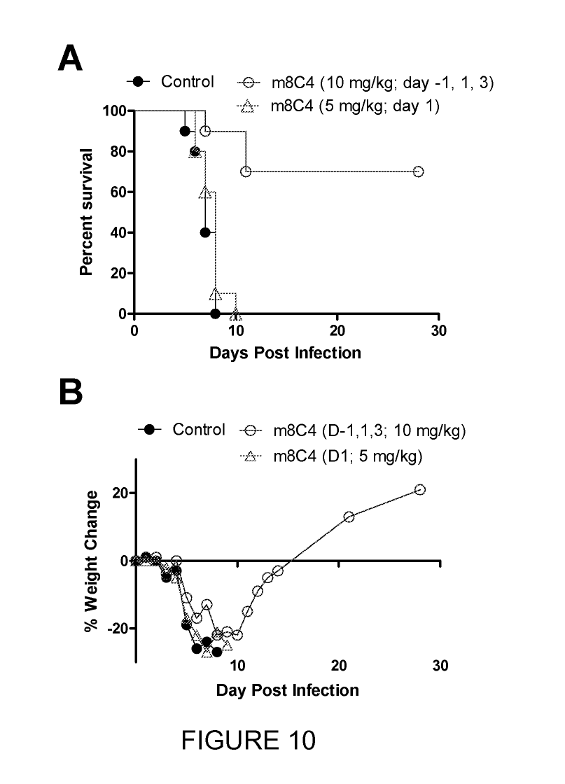

A lethal mouse model for SUDV has been recently developed using mice lacking receptors for IFNα and IFNβ (IFNαβ^{-/-}) (Brannan, J. M., et al., J Infect Dis, jiv215. [Epub ahead of print] (2015)). Since m8C4 showed the strong neutralization toward SUDV, we tested this antibody in the SUDV mouse model. Groups of 10 mice were infected with 1000 PFU of wild type SUDV. One group received m8C4 (10 mg/kg) 24 h before as well as 24h and 72h after infection, while a second group received m8C4 (5 mg/kg) only on day 1 post infection. While all control mice and mice receiving a single dose of 5 mg/kg m8C4 succumbed to infection, treatment with three doses of m8C4 led to 70% protection (FIG. 10A-B). Thus, m8C4 represents the prototype of an antibody with cross neutralization and protective efficacy against two widely divergent ebolavirus species.

D. Discussion

In this example we show that a single antibody (m8C4) can exhibit neutralization and protective efficacy against two different filoviruses, EBOV and SUDV, which are the most divergent ebolaviruses, sharing only 56% sequence identity within GP. Other available SUDV mAb immunotherapeutic candidates include 16F6 and its synthetic, human analogs E10 and F4, which confer protection and memory immunity in the SUDV murine model (Dias, J. M., et al., Nat Struct Mol Biol 18, 1424-1427 (2011); Chen, G., et al., ACS Chem Biol 9, 2263-2273 (2014)). However, these SUDV mAbs are strictly species-specific and bind at a distinct epitope at the base of pre-fusion GP2 (Dias, J. M., et al.). m8C4 binds a different protective epitope for SUDV GP.

Another mAb described here, m4B8, bound with high affinity to EBOV, SUDV, BDBV, and RESTV and provided 100% efficacy upon two injections in a mouse model of EBOV infection. Single delivery of this mAb on day 3 (peak of viremia in mice) also provided protection. While m16G8 and m17C6 alone provided low level of protection in mice, a cocktail of m8C4 and m16G8, displayed synergy when combined at lower doses. The only pan-filovirus mAb identified in this screen (m21D10 bound with low affinity to all filoviruses tested but failed to protect against lethal challenge.

Synergy between m8C4 and m16G8 in murine challenge studies was observed even though both these mAbs bound GP with low to moderate affinity. This synergy between a glycan cap and a potential GP2 binder demonstrates that greater efficacy can be achieved by targeting multiple epitopes within ebolavirus GP domains.

### Example 5: Targeting of Antibodies to Endosomal Compartment

Productive entry of enveloped viruses into the susceptible host cells requires fusion of the viral envelope with the host cell plasma membrane. In most instances upon internalization the virus is delivered into the endosomal compartments in which the pH-dependent membrane fusion is initiated by the envelope glycoprotein (Hunt, et al., 2012, Viruses, 4 (2):258-275; Luo, 2012, Adv Exp Med Biol, 726:201-221; Modis, 2013, Adv Exp Med Biol, 790:150-166; Rojek and Kunz, 2008, Cell Microbiol, 10 (4):828-835). The interaction of the viral glycoprotein with its cellular entry receptor can occur on the cell surface or be an endosomal event secondary to the initial uptake of the virus. Viruses that are internalized via receptor-mediated endocytosis interact with their receptor on the cell surface and usually the same receptor that co-migrates with the virus into the endosome also mediates membrane fusion and delivery into the cytoplasm (Grove and Marsh, 2011, J Cell Biol, 195 (7):1071-1082). Alternatively the fusion can occur at the plasma membrane (Grove and Marsh, 2011, J Cell Biol, 195 (7):1071-1082; Marechal, et al., 2001, J Virol, 75 (22):11166-11177). However, viruses can also utilize macropinocytosis as a cell-type specific, receptor independent endocytic pathway. Recent studies suggest that filoviruses EBOV and MARV use macropinocytosis as the primary mechanism of entry into the cells (Hunt, et al., 2011, J Virol, 85 (1):334-347; Nanbo, et al., 2010, PLoS Pathog, 6 (9):e1001121; Saeed, et al., 2010, PLoS Pathog, 6 (9):e1001110). The macropinocytic vesicles deliver the virus to the endosomes where, at the low pH, the productive interaction of the viral glycoprotein with its receptor, primarily NPC-1 (Carette, et al., 2011, Nature, 477 (7364):340-343) leads to membrane fusion.

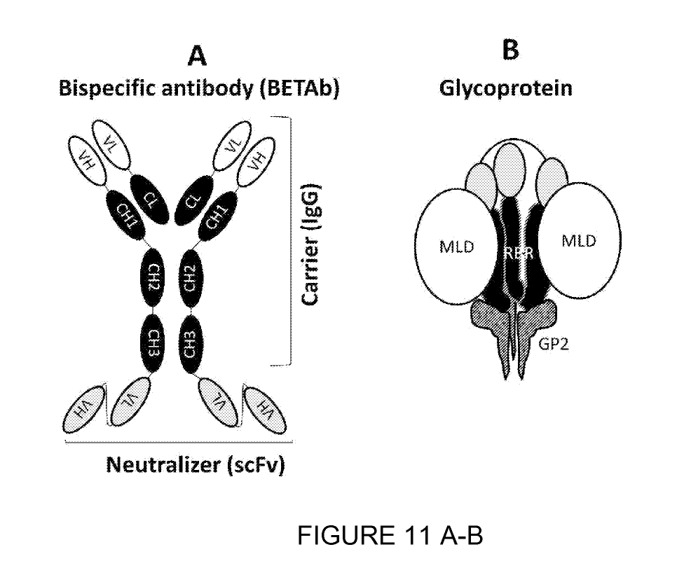

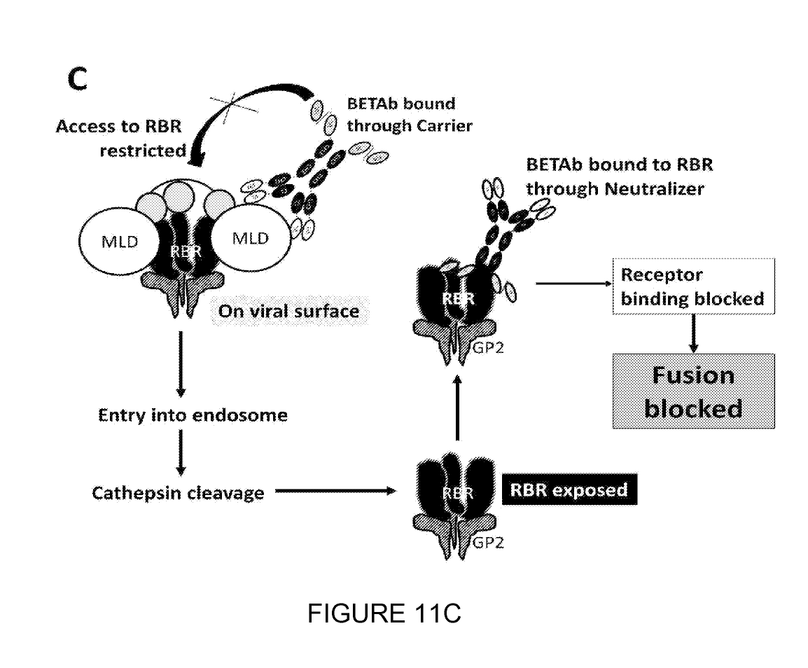

The receptor binding region (RBR) of the filoviruses has been shown to encompass about 150 amino acids located at the N-terminus of the GP-1 subunit of the glycoprotein (Kuhn, et al., 2006, J Biol Chem, 281 (23):15951-15958, see also FIG. 4A). The crystal structure of the EBOV glycoprotein demonstrates that the RBR is largely shielded by a large, highly glycosylated mucin-like domain as well as a smaller structure called the glycan cap. These domains restrict access to the receptor binding regions (Lee, et al., 2008, Nature, 454 (7201):177-182) (illustrated in FIG. 11B). However, once the virus enters the endosome, the cellular cathepsins cleave the viral glycoprotein at a site between the RBR and the glycan cap leaving naked RBR attached to the GP2 fusion domain (Chandran, et al., 2005, Science, 308 (5728):1643-1645a; Kaletsky, et al., 2007, J Virol, 81 (24):13378-13384). In this cleaved protein the RBR is fully exposed and can now effectively interact with the receptor. A major obstacle in developing neutralizing antibodies for filoviruses is the fact that antibodies that recognize the RBR can have reduced access their epitopes due to steric masking of the RBR. This example provides an approach for targeting anti-RBR antibodies to the endosomes where the antibodies can gain access to the RBR and can neutralize the virus by occupying the receptor binding sites.

As illustrated in FIG. 11A, a bispecific endosome-targeted antibody (BETAb) is constructed comprising a first binding domain and a second binding domain. The first binding domain, the “neutralizer binding domain,” specifically binds to a viral receptor binding region or other secluded region of the virus surface involved in entry into a host cell's cytoplasm, e.g., a region of a surface glycoprotein of a virus that enters cells through membrane fusion events in the endosome. The second binding domain, “the carrier binding domain,” specifically binds to a target epitope well exposed on the surface of the same virus. For a filovirus BETAb, the neutralizer binding domain can be an antibody or antigen-binding fragment thereof, e.g., a ScFv or an ScFab, which specifically binds to the RBR region of the GP-1 subunit. Exemplary, non-limiting antibodies suitable as neutralizer binding domains include m2D8, m21D10, m5E4, m16G8, m17C6, m8C4, m4B8, m21B2, m2E4, or any fragment, variant, or derivative thereof, or any combination thereof. For a filovirus BETAb, the carrier binding domain can be an antibody or antigen-binding fragment thereof, e.g., a ScFv or an ScFab, which specifically binds to a target epitope including, but not limited to an epitope located in the mucin-like domain, an epitope located in the glycan cap, an epitope located in the GP2 fusion domain, or any combination thereof. Upon endocytosis or macropinocytosis of the virus by the host cell, this bispecific endosome-targeted antibody (BETAb), bound to the virus via the carrier binding domain, will co-migrate into the endosomes. Upon proteolysis of the glycoprotein (as in the case of Ebola virus), or pH-dependent conformational changes of the glycoprotein, the neutralizer binding domain of the bispecific antibody can bind to the glycoprotein, thus blocking the receptor binding sites and preventing virus entry into the cytoplasm. Such bispecific antibodies can be produced for filoviruses, e.g., ebolavirus, Marburg virus, or sudan virus, or for any virus that enters host cells through fusion events in the endosomes. Examples of such viruses include but are not limited to, influenza virus, Dengue virus, hepatitis C virus, metapneumovirus, arenaviruses, and alphaviruses.

FIG. 11A illustrates one non-limiting form of a prototypic BETAb, and various molecular approaches can be applied for linking the neutralizer and carrier and generation of bispecific endosome-targeted antibodies, including, without limitation:
A single-chain Fv (Sc-FV) version of the neutralizer antibody can be fused to one of the four termini of the full-length carrier antibody (N and C termini of the heavy or light chain).
A single-chain Fv (Sc-FV) version of the carrier antibody can be fused to one of the four termini of the full-length neutralizer antibody (N and C termini of the heavy or light chain).
A single-chain Fv (Sc-FV) version of the neutralizer antibody can be fused to one of the two termini of a single-chain Fv (Sc-FV) version of the carrier antibody (N and C termini of the scFv).
A single-chain Fv (Sc-FV) version of the carrier antibody can be fused to one of the two termini of a single-chain Fv (Sc-FV) version of the neutralizer antibody (N and C termini of the scFv).
A single chain Fab (scFab) fragment of each antibody can be generated by linking (through a flexible linker) the full length light chain (variable and constant domains) to a truncated form of the heavy chain of the same antibody containing the variable region and the CH1 domain. The scFab of the neutralizer antibody is then genetically fused to a scFab of the carrier antibody at either N or C termini.

In all above examples scFab, scFv, or other antibodies or antigen-binding fragments thereof can be used interchangeably.

Tethering of the neutralizer and carrier by any other technology, including chemical conjugation, with the intent of targeting the bispecific antibody along with the targeted virus into the endosomes.

The BETAb can be expressed in a mammalian cell expression system, a plant expression system, or an insect cell expression system, or a prokaryotic expression system.

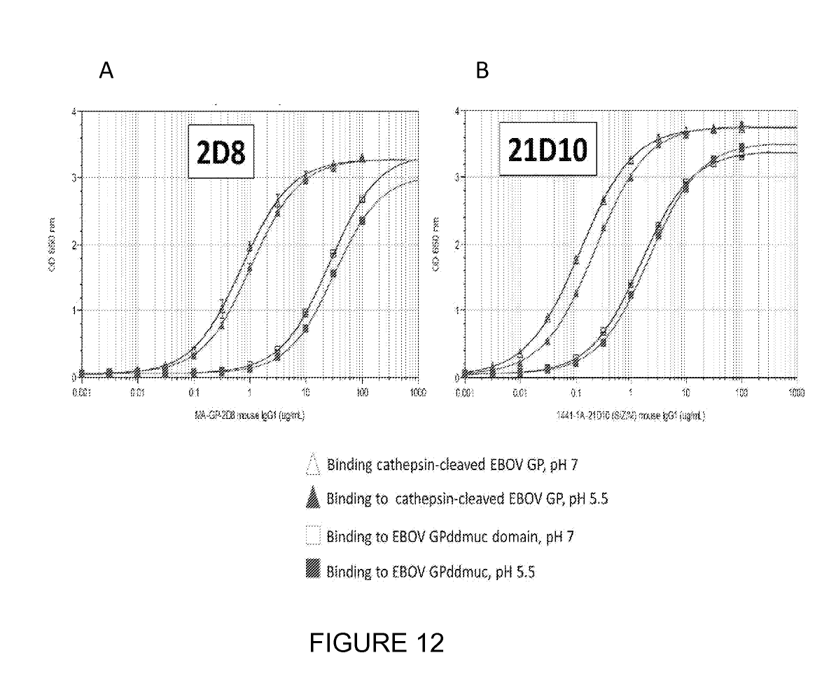

In certain aspects, the “Neutralizer” antibody component of the BETAb can bind to its target at acidic environment of endosomes (pH 5.5). At least two RBR-binding antibodies disclosed herein, m2D8 (SEQ ID NOs: 1-10) and m21D10 (SEQ ID NOs: 11-20) demonstrate this property. FIGS. 12A-B show the binding of m2D8 and m21D10 to EBOV glycoprotein at neutral and acidic pH as an example. This figure further demonstrates enhanced binding of the antibody to GP when the bulky MLD domain is deleted.

The breadth and scope of the present disclosure should not be limited by any of the above-described exemplary embodiments, but should be defined only in accordance with the following claims and their equivalents.

What is claimed is:

1. An isolated binding molecule or antigen-binding fragment thereof comprising a first binding domain that specifically binds to an orthologous filovirus glycoprotein epitope, wherein the binding domain specifically binds to the epitope on two or more filovirus species or strains.

2. The binding molecule or fragment thereof of claim 1, wherein the first binding domain can specifically bind to the orthologous epitope as expressed in two or more, three or more, four or more, or five or more of Marburg virus (MARV), Ravn virus (RAVV), Tai Forest virus (TAFV), Reston virus (RESTV), Sudan virus (SUDV), Ebola virus (EBOV), and Bundibugyo virus (BDBV).

3. The binding molecule or fragment thereof of claim in claim 2, wherein the first binding domain can bind to the orthologous epitope as expressed in two or more, three or more, four or more, or all five of EBOV, SUDV, MARV, RESTV, and BDBV.

4. The binding molecule or fragment thereof of any one of claims 1 to 3, wherein the orthologous epitope is in the receptor-binding region (RBR) of GP-1 subunit of the viral glycoprotein.

5. The binding molecule or fragment thereof of any one of claims 1 to 4, wherein the first binding domain can bind to the orthologous epitope as expressed in EBOV, SUDV, MARV, RESTV, and BDBV.

6. The binding molecule or fragment thereof of claim 5, wherein the first binding domain can bind to the same orthologous epitope as an antibody or antigen-binding fragment thereof comprising a heavy chain variable region (VH) and a light chain variable region (VL) comprising, respectively, the amino acid sequences SEQ ID NO: 2 and 7, or SEQ ID NO: 12 and 17.

7. The binding molecule or fragment thereof of claim 5, wherein the first binding domain can competitively inhibit antigen binding by an antibody or antigen-binding fragment thereof comprising a VH and a VL comprising, respectively, the amino acid sequences SEQ ID NO: 2 and 7, or SEQ ID NO: 12 and 17.

8. The binding molecule of claim 6 or claim 7, wherein the first binding domain can bind to an orthologous epitope within the amino acid sequence SEQ ID NO: 109.

9. The binding molecule of claim 6 or claim 7, wherein the first binding domain can bind to an orthologous epitope within the amino acid sequence SEQ ID NO: 110.

10. The binding molecule or fragment thereof of any one of claims 1 to 3, wherein the first binding domain can bind to the orthologous epitope as expressed in at least EBOV, SUDV, and MARV.

11. The binding molecule or fragment thereof of claim 10, wherein the first binding domain can bind to the same orthologous epitope as an antibody or antigen-binding fragment thereof comprising a VH and a VL comprising the amino acid sequences SEQ ID NO: 22 and 27.

12. The binding molecule or fragment thereof of claim 10, wherein the first binding domain can competitively inhibit antigen binding by an antibody or antigen-binding fragment thereof comprising a VH and a VL comprising the amino acid sequences SEQ ID NO: 22 and 27.

13. The binding molecule or fragment thereof of any one of claims 1 to 3, wherein the first binding domain can bind to the orthologous epitope as expressed in EBOV, SUDV, RESTV, and BDBV.

14. The binding molecule or fragment thereof of claim 13, wherein the first binding domain can bind to the same orthologous epitope as an antibody or antigen-binding fragment thereof comprising a VH and a VL comprising, respectively, the amino acid sequences SEQ ID NO: 32 and 37, SEQ ID NO: 42 and 47, or SEQ ID NO: 62 and 67.

15. The binding molecule or fragment thereof of claim 13, wherein the first binding domain can competitively inhibit antigen binding by an antibody or antigen-binding fragment thereof comprising a VH and a VL comprising, respectively, the amino acid sequences SEQ ID NO: 32 and 37, SEQ ID NO: 42 and 47, or SEQ ID NO: 62 and 67.

16. The binding molecule or fragment thereof of any one of claims 1 to 3, wherein the first binding domain can bind to the orthologous epitope as expressed in EBOV, SUDV and BDBV.

17. The binding molecule or fragment thereof of claim 16, wherein the first binding domain can bind to the same orthologous epitope as an antibody or antigen-binding fragment thereof comprising a VH and a VL comprising the amino acid sequences SEQ ID NO: 52 and 57.

18. The binding molecule or fragment thereof of claim 16, wherein the first binding domain can competitively inhibit antigen binding by an antibody or antigen-binding fragment thereof comprising a VH and a VL comprising the amino acid sequences SEQ ID NO: 52 and 57.

19. The binding molecule or fragment thereof of any one of claims 1 to 3, wherein the first binding domain can bind to the orthologous epitope as expressed in SUDV and MARV.

20. The binding molecule or fragment thereof of claim 19, wherein the first binding domain can bind to the same orthologous epitope as an antibody or antigen-binding fragment thereof comprising a VH and a VL comprising the amino acid sequences SEQ ID NO: 72 and 77.

21. The binding molecule or fragment thereof of claim 19, wherein the first binding domain can competitively inhibit antigen binding by an antibody or antigen-binding fragment thereof comprising a VH and a VL comprising the amino acid sequences SEQ ID NO: 72 and 77.

22. The binding molecule or fragment thereof of any one of claims 1 to 3, wherein the first binding domain can bind to the orthologous epitope as expressed in EBOV, SUDV, and RESTV.

23. The binding molecule or fragment thereof of claim 22, wherein the first binding domain can bind to the same orthologous epitope as an antibody or antigen-binding fragment thereof comprising a VH and a VL comprising the amino acid sequences SEQ ID NO: 82 and 87.

24. The binding molecule or fragment thereof of claim 22, wherein the first binding domain can competitively inhibit antigen binding by an antibody or antigen-binding fragment thereof comprising a VH and a VL comprising the amino acid sequences SEQ ID NO: 82 and 87.

25. The binding molecule of any one of claims 1 to 24, wherein the first binding domain can bind to the orthologous epitope in solution at a pH of about 4.0, about 4.5, about 5.0, about 5.5, about 6.0, about 6.5, about 7.0, or about 7.5.

26. The binding molecule or fragment thereof of any one of claims 1 to 25, which comprises an antibody or antigen-binding fragment thereof.

27. The antibody or fragment thereof of claim 26, wherein the first binding domain comprises VL-CDR1, VL-CDR2, VL-CDR3, VH-CDR1, VH-CDR2, and VH-CDR3 amino acid sequences identical or identical except for four, three, two, or one single amino acid substitutions, deletions, or insertions in one or more CDRs to: SEQ ID NOs: 3, 4, 5, 8, 9, and 10; SEQ ID NOs: 13, 14, 15, 18, 19, and 20; SEQ ID NOs: 23, 24, 25, 28, 29, and 30; SEQ ID NOs: 33, 34, 35, 38, 39, and 40; SEQ ID NOs: 43, 44, 45, 48, 49, and 50; SEQ ID NOs: 53, 54, 55, 58, 59, and 60; SEQ ID NOs: 63, 64, 65, 68, 69, and 70; SEQ ID NOs: 73, 74, 75, 78, 79, and 80; or SEQ ID NOs: 83, 84, 85, 88, 89, and 90; respectively.

28. The antibody or fragment thereof of claim 26, wherein the first binding domain comprises VH and VL amino acid sequences at least 85%, 90%, 95%, or 100% identical to reference amino acid sequences SEQ ID NO: 2 and SEQ ID NO: 7; SEQ ID NO: 12 and SEQ ID NO: 17; SEQ ID NO: 22 and SEQ ID NO: 27; SEQ ID NO: 32 and SEQ ID NO: 37; SEQ ID NO: 42 and SEQ ID NO: 47; SEQ ID NO: 52 and SEQ ID NO: 57; SEQ ID NO: 62 and SEQ ID NO: 67; SEQ ID NO: 72 and SEQ ID NO: 77; or SEQ ID NO: 82 and SEQ ID NO: 87; respectively.

29. The antibody or fragment thereof of any one of claims 26 to 28, which is a human antibody, a murine antibody, a humanized antibody, a chimeric antibody, or a fragment thereof.

30. The antibody or fragment thereof of claim 29, which is a monoclonal antibody, a component of a polyclonal antibody mixture, a recombinant antibody, a multispecific antibody, or any combination thereof.

31. The antibody or fragment thereof of claim 30, which is a bispecific antibody or fragment thereof further comprising a second binding domain.

32. The antibody or fragment thereof of claim 31, wherein the second binding domain can specifically bind to a filovirus epitope that is surface exposed and accessible to the second binding domain on a filovirus virion particle.

33. The antibody or fragment thereof of claim 31 or claim 32, wherein the second binding domain can specifically bind to the mucin-like domain, an epitope located in the glycan cap, an epitope located in the GP2 fusion domain, or any combination thereof.

34. The antibody or fragment thereof of any one of claims 26 to 33, which comprises a heavy chain constant region or fragment thereof.

35. The antibody or fragment thereof of claim 34, wherein the heavy chain constant region or fragment thereof is a murine constant region or fragment thereof.

36. The antibody or fragment thereof of claim 34, wherein the heavy chain constant region or fragment thereof is a human constant region or fragment thereof.

37. The antibody or fragment thereof of claim 36, wherein the heavy chain constant region or fragment thereof is an IgM, IgG, IgA, IgE, IgD, or IgY constant region or fragment thereof.

38. The antibody or fragment thereof of any one of claim 37, wherein the human IgG constant region or fragment thereof is a human IgG1, IgG2, IgG3, or IgG4 constant region or fragment thereof.

39. The antibody or fragment thereof of any one of claims 26 to 38, further comprising a light chain constant region or fragment thereof.

40. The antibody or fragment thereof of claim 39, wherein the light chain constant region or fragment thereof is a murine constant region or fragment thereof.

41. The antibody or fragment thereof of claim 40, wherein the light chain constant region or fragment thereof is a human constant region or fragment thereof.

42. The antibody or fragment thereof of claim 41, wherein the light chain constant region or fragment thereof is human kappa or lambda constant region or fragment thereof.

43. The antibody or fragment thereof of any one of claims 26 to 42, wherein the first binding domain comprises a full-size antibody comprising two heavy chains and two light chains.

44. The antibody or fragment thereof of any one of claims 26 to 43, wherein first binding domain comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

45. The antibody or fragment thereof of any one of claims 31 to 33, wherein the second binding domain comprises a full-size antibody comprising two heavy chains and two light chains.

46. The antibody or fragment thereof of any one of claims 31 to 33, wherein second binding domain comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

47. The antibody or fragment thereof of any one of claims 26 to 46, wherein binding of the first binding domain to the orthologous epitope on a filovirus fully or partially neutralizes infectivity of the filovirus.

48. The antibody or fragment thereof of any one of claims 26 to 47, which is conjugated to an antiviral agent, a protein, a lipid, a detectable label, a polymer, or any combination thereof.

49. A composition comprising the antibody or fragment thereof of any one of claims 26 to 48, and a carrier.

50. A kit, comprising
(a) the antibody or antigen binding fragment thereof of any one of claims 26 to 48 or the composition of claim 49;
(b) instructions for using the antibody or fragment thereof or using the composition or directions for obtaining instructions for using the antibody or fragment thereof or using the composition.

51. The kit of claim 50, further comprising a buffer, a solid support, or both.

52. The kit of claim 51, wherein the solid support is a bead, a filter, a membrane or a multiwall plate.

53. The kit of claim 51, wherein the buffer is suitable for an enzyme-linked immunosorbent assay (ELISA).

54. An isolated polynucleotide comprising a nucleic acid encoding the binding molecule or fragment thereof of any one of claims 1 to 25 or a subunit thereof, or the antibody or fragment thereof of any one of claims 26 to 48; or a subunit thereof.

55. The polynucleotide of claim 54, wherein the nucleic acid encodes a VH, and wherein the VH comprises VH-CDR1, VH-CDR2, and VH-CDR3, wherein the VH-CDRs comprise, respectively, amino acid sequences identical to, or identical except for four, three, two, or one single amino acid substitutions, deletions, or insertions in one or more of the VH-CDRs to: SEQ ID NOs: 3, 4, and 5; SEQ ID NOs: 13, 14, and 15; SEQ ID NOs: 23, 24, and 25; SEQ ID NOs: 33, 34, and 35; SEQ ID NOs: 43, 44, and 45; SEQ ID NOs: 53, 54, and 55; SEQ ID NOs: 63, 64, and 65; SEQ ID NOs: 73, 74, and 75; or SEQ ID NOs: 83, 84, and 85.

56. The polynucleotide of claim 54, wherein the nucleic acid encodes a VL, and wherein the VL comprises a VL-CDR1, a VL-CDR2, and a VL-CDR3, wherein the VL-CDRs comprise, respectively, amino acid sequences identical to, or identical except for four, three, two, or one single amino acid substitutions, deletions, or insertions in one or more of the VL-CDRs to: SEQ ID NOs: 8, 9, and 10; SEQ ID NOs: 18, 19, and 20; SEQ ID NOs: 28, 29, and 30; SEQ ID NOs: 38, 39, and 40; SEQ ID NOs: 48, 49, and 50; SEQ ID NOs: 58, 59, and 60; SEQ ID NOs: 68, 69, and 70; SEQ ID NOs: 78, 79, and 80; or SEQ ID NOs: 88, 89, and 90.

57. The polynucleotide of claim 54, wherein the nucleic acid encodes a VH, and wherein the VH comprises an amino acid sequence at least 85%, 90%, 95%, or 100% identical to the reference amino acid sequence SEQ ID NO: 2; SEQ ID NO: 12; SEQ ID NO: 22; SEQ ID NO: 32; SEQ ID NO: 42; SEQ ID NO: 52; SEQ ID NO: 62; SEQ ID NO: 72; or SEQ ID NO: 82.

58. The polynucleotide of claim 54, wherein the nucleic acid encodes a VL, and wherein the VL comprises an amino acid sequence at least 85%, 90%, 95%, or 100% identical to the reference amino acid sequence SEQ ID NO: 7; SEQ ID NO: 17; SEQ ID NO: 27; SEQ ID NO: 37; SEQ ID NO: 47; SEQ ID NO: 57; SEQ ID NO: 67; SEQ ID NO: 77; or SEQ ID NO: 87.

59. A vector comprising the polynucleotide of any one of claims 54 to 58.

60. A composition comprising the polynucleotide of any one of claims 54 to 58 or the vector of claim 59.

61. A polynucleotide or a combination of polynucleotides encoding the binding molecule or fragment thereof of any one of claims 1 to 25 or the antibody or fragment thereof of any one of claims 26 to 48.

62. The polynucleotide or combination of polynucleotides of claim 61, comprising a nucleic acid encoding a VH, and a nucleic acid encoding a VL, wherein the VH and VL comprise VL-CDR1, VL-CDR2, VL-CDR3, VH-CDR1, VH-CDR2, and VH-CDR3 amino acid sequences identical or identical except for four, three, two, or one single amino acid substitutions, deletions, or insertions in one or more CDRs to: SEQ ID NOs: 3, 4, 5, 8, 9, and 10; SEQ ID NOs: 13, 14, 15, 18, 19, and 20; SEQ ID NOs: 23, 24, 25, 28, 29, and 30; SEQ ID NOs: 33, 34, 35, 38, 39, and 40; SEQ ID NOs: 43, 44, 45, 48, 49, and 50; SEQ ID NOs: 53, 54, 55, 58, 59, and 60; SEQ ID NOs: 63, 64, 65, 68, 69, and 70; SEQ ID NOs: 73, 74, 75, 78, 79, and 80; or SEQ ID NOs: 83, 84, 85, 88, 89, and 90; respectively.

63. The polynucleotide or combination of polynucleotides of claim 61, comprising a nucleic acid encoding a VH, and a nucleic acid encoding a VL, wherein the VH and VL comprise amino acid sequences at least 85%, 90%, 95%, or 100% identical to reference amino acid sequences selected from the group consisting of SEQ ID NO: 2 and SEQ ID NO: 7; SEQ ID NO: 12 and SEQ ID NO: 17; SEQ ID NO: 22 and SEQ ID NO: 27; SEQ ID NO: 32 and SEQ ID NO: 37; SEQ ID NO: 42 and SEQ ID NO: 47; SEQ ID NO: 52 and SEQ ID NO: 57; SEQ ID NO: 62 and SEQ ID NO: 67; SEQ ID NO: 72 and SEQ ID NO: 77; or SEQ ID NO: 82 and SEQ ID NO: 87; respectively.

64. The polynucleotide or combination of polynucleotides of any one of claims 61 to 63, wherein the nucleic acid encoding a VH and the nucleic acid encoding a VL are in the same vector.

65. The vector comprising the polynucleotide or combination of polynucleotides of claim 64.

66. The polynucleotide or combination of polynucleotides of any one of claims 61 to 63, wherein the nucleic acid encoding a VH and the nucleic acid encoding a VL are in different vectors.

67. The vectors comprising the polynucleotide or combination of polynucleotides of claim 66.

68. A host cell comprising the polynucleotide or combination of polynucleotides of any one of claims 54 to 58 or 61 to 63 or the vector or vectors of any one of claim 59, 65 or 67.

69. A method of making the binding molecule or fragment thereof of any one of claims 1 to 25 or the antibody or fragment thereof of any one of claims 26 to 48, comprising
(a) culturing the cell of claim 68; and
(b) isolating the binding molecule or fragment thereof or antibody or fragment thereof.

70. A diagnostic reagent comprising the binding molecule or fragment thereof of any one of claims 1 to 25 or the antibody or fragment of any one of claims 26 to 48.

71. A method for preventing, treating, or managing filovirus infection in a subject, comprising administering to a subject in need thereof an effective amount of the antibody or antigen binding fragment thereof of any one of claims 26 to 48 or the composition of claim 49.

72. The method of claim 71, wherein the filovirus is MARV, RAVV, TAFV, RESTV, SUDV, EBOV, BDBV, or any combination thereof.

73. The method of claim 71 or claim 72, wherein the filovirus infection is hemorrhagic fever.

74. The method of any one of claims 71 to 73, wherein the subject is a nonhuman primate or a human.

75. A method of neutralizing a virus that enters host cells through fusion events in the host cell endosomes, comprising contacting the virus with a bispecific antibody comprising a first binding domain and a second binding domain, wherein the first binding domain binds to a epitope of the virus that interacts with a host cell surface receptor, and the second binding domain binds to an epitope on the surface of the intact virus, wherein the virus/antibody complex is inhibited from fusing with the host cell membrane in the endosome, thereby neutralizing the virus.
